(19) United States
(12) Patent Application Publication
(10) Pub. No.: US 2004/0120990 A1
(43) Pub. Date: Jun. 24, 2004

(54) Absorbent proteins and methods for using same

(76) Inventors: John C. Cushman, Reno, NV (US); Christina Walters, Ft. Collins, CO (US)

Correspondence Address: NATH & ASSOCIATES, 6th Floor, 1030 15th Street, N.W., Washington, DC, 20005, US

(21) Appl. No.: 10/637,706

(22) Filed: Aug. 11, 2003

Related U.S. Application Data
(60) Provisional application No. 60/403,329, filed on Aug. 12, 2002.

(51) Int. Cl. A61K 9/70

(52) U.S. Cl. 424/443

(57) ABSTRACT

The present inventive subject matter relates to novel methods for using water-absorbing and water-retaining LEA Group 1 proteins. Addition of water to an LEA Group 1 protein produces a hydrogel, which is useful in absorbent materials and compositions; as a therapeutic for skin; as a pharmaceutical or cosmetic excipient; in food applications where their hydrophilic properties improve moisture absorption and retention, and reduce the formation of ice crystals upon freezing; as cryoprotectants for maintaining the integrity of biologically relevant molecules upon freezing; and for increasing the resistence of an organism to drought stress, osmotic stress, heat stress, freezing stress, or a combination thereof.

This application claims the benefit of U.S. Provisional Patent Application No. 60/403,329, filed Aug. 12, 2002, the contents of which is hereby incorporated by reference in its entirety.

This work was funded by United States Department of Agriculture Grant No. 1998-35100-10216. The U.S. government may have rights in the inventive subject matter by virtue of this support.

### BACKGROUND OF THE INVENTION

1. Field of the Invention

The present inventive subject matter relates to novel methods for using water-absorbing and water-retaining LEA Group 1 proteins. Addition of water to an LEA Group 1 protein produces a hydrogel, which is useful in absorbent materials and compositions; as a therapeutic for skin; as a pharmaceutical or cosmetic excipient; in food applications where their hydrophilic properties improve moisture absorption and retention, and reduce the formation of ice crystals upon freezing; as cryoprotectants for maintaining the integrity of biologically relevant molecules upon freezing; and for increasing the resistence of an organism to drought stress, osmotic stress, heat stress, freezing stress, or a combination thereof.

2. Background

Absorbent materials. A variety of absorbent materials are used in exemplary products such as paper towels, moist towelettes, tissues, and toilet paper, as well as in personal hygiene products which absorb body fluids such as urine, menses, and wound exudate. Such absorbent materials are generally placed on or near the material to be absorbed to serve these purposes.

Paper towels, moist towelettes, tissues, and toilet paper have become genericized products. Many consumers select such products by price, or by special features such as absorbency or strength.

One important class of personal hygiene products includes diapers, where the absorbent material can be derived from wood pulp, cellulosic fibers, or super absorbent, synthetically produced material. Diapers commonly have an inner core designed to absorb urine and water. The core is typically formed from a superabsorbent polymer dispersed in a larger amount of less absorbent material. The absorbent materials typically contained in the core are separated from the skin by at least one layer of material. The absorbent materials absorb urine and can become saturated. It is believed that some material from the absorbent core leaches from the wet absorbent and travels back to the skin. In the case of chemically treated absorbent materials and films, the leachate may be irritating and thus not beneficial. Further, skin contact with urine can result in irritation, which in turn may exacerbate diaper rash problems.

Other products which contain absorbent materials include feminine hygiene products, such as tampons and pads, and wound dressings for humans or animals. For specific applications, wound dressings preferably absorb exudate from wounds while keeping the wounds relatively moist to promote healing. In some applications, a gel may be desirable as a wound dressing, where the gel can maintain a moist wound environment, while absorbing excess exudate.

Hydrogels.

The ability to provide highly absorbent materials in articles such as diapers has been contingent on the ability to develop absorbent cores or structures that can acquire and store large quantities of discharged body liquids, in particular urine. In this regard, the use of certain absorbent polymers, often referred to as hydrogels, superabsorbents, or hydrocolloid material has been particularly important (see, for example, U.S. Pat. No. 3,699,103 to Harper et al., issued Jun. 13, 1972, and U.S. Pat. No. 3,670,731 to Harmon, issued Jun. 20, 1972, which disclose the use of hydrogel-forming absorbent polymers in absorbent articles.) U.S. Pat. No. 4,673,402 to Weisman, et al., issued Jun. 16, 1987 and U.S. Pat. No. 4,935,022 to Lash et al., issued Jun. 19, 1990, disclose dual-layer core structures comprising a fibrous matrix and hydrogel-forming absorbent polymers useful in fashioning thin, compact, nonbulky diapers. U.S. Pat. Nos. 5,562,646 and 5,599,335 to Goldman, et al., issued Oct. 8, 1996 and Feb. 4, 1997, respectively, each relates to absorbent cores comprising regions of high concentrations of hydrogel-forming polymer, where the polymer forms a gel-continuous liquid transportation zone upon swelling.

Starch-based Superabsorbents.

Polymeric substances which possess the ability to absorb aqueous fluids are known in the prior art. For example, U.S. Pat. Nos. 3,669,103 and 3,810,468 disclose that a variety of monomers may be polymerized, with crosslinking, to give polymeric absorbents. The crosslinking reaction is of critical importance, since the non-crosslinked polymers are water soluble and thus have no utility as absorbents.

U.S. Pat. No. 3,661,815 discloses water-absorbing alkali metal salts of saponified granular starch-PAN graft copolymers.

U.S. Pat. No. 3,932,322 discloses a mixture of the composition of U.S. Pat. No. 3,661,815 with fumed silica or alumina. This mixture exhibits an increased rate of fluid uptake and a decreased tendency toward dusting.

Water-absorbing alkali metal salts of saponified gelatinized starch-PAN graft copolymers are disclosed in U.S. Pat. No. 3,935,099. Contrary to the absorbent composition of U.S. Pat. No. 3,661,815, the composition of U.S. Pat. No. 3,935,099 may be dried to a continuous film which has an unusually high absorbency for aqueous fluids. Moreover, this film-forming tendency permits a variety of substrates to be coated with thin films of the absorbent composition and thus leads to dramatic increases in fluid absorbencies of the substrates.

U.S. Pat. No. 4,045,387 discloses highly absorbent polymeric compositions prepared by essentially the same process disclosed in U.S. Pat. No. 3,935,099, except that flour is substituted for the starch. These flour-derived absorbents have higher water absorbencies than the corresponding products derived from starch.

U.S. Pat. No. 4,194,998 discloses highly absorbent polyhydroxy polymer graft copolymers of starch prepared by a simplified method of synthesis using a mixture of a nonionic acrylic monomer and an anionic sulfonic acid-substituted acrylic monomer, without the need for an alkaline saponification step. These compositions are also characterized by their ability to absorb large amounts of aqueous fluids under highly acidic conditions.

U.S. Pat. No. 4,483,950 discloses starch-based superabsorbents which are extended by blending with highly modified, low molecular weight dextrinized starches which synergistically interact with the superabsorbents, thereby permitting dilution without a commensurate reduction in water absorbency. While the principal utility of the blends is the absorption of aqueous fluids, when hydrated they yield soft, smooth gels useful as high-quality thickening agents.

Polymer Matrices and Resins.

Polymer matrices or foams are formed by introducing a gas into a solution of hydrogel monomers during the polymerization of the monomers. Hydrogel foams have numerous gas cells, the majority of which are connected to form open channel systems. The size and number of the gas cells is determined by monomer concentration, the viscosity of the monomer solution, the extent of crosslinking, the presence/type of surfactant and the type and amount of gas introduced into the polymerizing solution. Polymeric foams are produced from materials such as polyurethanes, rubber, and poly(vinyl chloride). The key ingredient in the foaming process is a blowing agent or foaming agent, which is defined as any substance or combination of substances capable of producing cellular structure within a polymer matrix.

Water-absorbent resins have been widely used in hygienic materials such as sanitary materials, paper diapers, water retainers for soil, and the like. Water insoluble crosslinked polymers, also known as water-absorbent resins, include crosslinked polyacrylic acid salts, self-crosslinking type polyacrylic acid salts, and crosslinked copolymer of starch-grafted acrylic acid salts.

In the manufacture of absorbent articles, it is desirable to employ materials having high void volume, a hydrophilic nature, and wet resiliency, or the ability to maintain void volume when wet and when under load. Non-woven materials made from long synthetic fibers offer high void volume and wet resiliency, but lack inherent hydrophilicity. The fibers can be treated with surfactants or other additives to increase the hydrophilicity of the structure, but such processes are expensive and do not provide the same level of affinity for water offered by cellulose or other hydrophilic polymers. Open cell plastics or plastic foams, also referred to as expanded or sponge plastics, can offer very high void volume and wet resiliency, but suffer the same problem of lacking inherent hydrophilicity and have high cost. Foams made of hydrophilic materials, such as superabsorbent foams, offer the ability to absorb and retain liquids, but can suffer from poor wet resiliency or high cost. Fiber reinforced foams are known wherein fibers are added to increase the strength of a foam matrix, but such materials are generally hydrophobic and lack the high-bulk, absorbent attributes desired of an absorbent article.

The general state-of-the-art relating to the uses for absorbent materials is both broad and varied, and is generally described in the following exemplary U.S. patents.

U.S. Pat. No. 5,750,585 to Park, et al., issued May 12, 1998, discloses a water swellable foam matrix formed as a macroporous solid, comprising a foam stabilizing agent and a polymer or copolymer of a free radical polymerizable hydrophilic olefin monomer crosslinked with about 0.1 to about 10% by weight of a multiolefin-functional crosslinking agent. The foam matrix is disclosed as being characterized by rapid swelling and high water swelling ratios.

U.S. Pat. No. 6,261,679 to Chen, et al., issued Jul. 17, 2001, discloses a fibrous absorbent structure that is wet stable and has large void volume with a density below the critical density of the fiber employed. The fibrous absorbent is disclosed to have several embodiments, including open-celled foam technologies to keep the fibrous structure expanded and bonded, open-celled polymeric foam with fibers serving as struts stabilized by binder material, and a fibrous structure filled with hydrophilic open-celled foams with the cell size substantially smaller than the fibrous pores. Disclosed uses include in a disposable product intended for the absorption of fluid such as body fluid, including extensible absorbent articles.

U.S. Pat. No. 6,303,711 to Sumiya, et al., issued Oct. 16, 2001, discloses a water-absorbent resin derived from monomer unit components comprising 40-99 % wt. of hydroxyl alkyl (meth)acrylate, 1-60% wt. of (meth)acrylic acid and/or alkali metal salt thereof, and 0.00001-3% wt. of a crosslinking agent. A second water-absorbent or water-retention material comprising water-swellable polymer is obtained by further thermally crosslinking particles of partially alkaline neutralized product of a copolymer of 20-99% mol of acrylamide and 1-80% mol of acrylic acid.

Other water-absorbent resins include a crosslinked polymer of sulfonic acid monomer or a crosslinked copolymer of sulfonic acid monomer with acrylic acid; a crosslinked copolymer of acrylic acid salt and poly (vinyl alcohol); and a crosslinked copolymer of acrylamide and acrylic acid.

U.S. Pat. No. 6,372,953 to Young, et al., issued Apr. 16, 2002, discloses absorbent members useful in the containment of body liquids such as urine, comprising at least one osmotic absorbent which is preferably a hydrogel-forming absorbent polymer and a high surface area material, and having a high capillary suction capacity.

More particularly, the state of the art relating to absorbent proteins is described in U.S. Pat. No. 6,544,548 to Siller-Jackson, et al., issued Apr. 8, 2003, which discloses keratin compositions and methods for making same. Specifically, Siller-Jackson discloses a hydratable keratin solid which forms a hydrogel upon addition of water, for use in various applications including non-woven films, diapers, skin treatments, prosthetic devices, excipients, tissue engineering scaffolds, and as an excipient for controlled drug delivery.

Preservation of Food and Other Biological Materials.

Biological materials are stored at reduced temperatures to decrease the rate of deterioration of the biological material. The low temperature inhibits the activity of degradation enzymes in the biological material, as well as inhibiting the growth of microorganisms which degrade the material. In addition to short-term storage at temperatures above freezing, freezing allows for storage for protracted periods and shipping over long distances.

When food products are frozen, ice-crystals are formed throughout the cells of many food products, often producing rupture of the cells. Thus, the formation of ice crystals within the food often results in damage to the food, which in turn reduces the quality of the food. In the process of freezing, the reduction in the quality of material stored in the frozen state also results in a reduction in the value of the material relative to the fresh, unfrozen state.

Further, when subjected to temperature changes during storage, as often occurs during storage in modern self-defrosting refrigerators, the shape and size of the initially formed crystals changes, often to the further detriment of the original texture and taste of the frozen food. Ice crystals often become larger and more moisture is removed from frozen food during longer storage intervals. Texture deterioration is due at least in part to the dehydration of the food product and is commonly referred to as “freezer burn.” Although most food storage packaging on the market today appears to be airtight, when stored for extended periods of time, air does penetrate the packaging, creating frost which in turn causes freezer burn.

In one approach to solving these problems, the conditions upon freezing are manipulated. Generally, an increase of the rate of freezing leads to a decrease in the aspect ratio for ice-crystals, and this characteristic has been exploited through the use of many quick freezing processes. Similarly, the presence or addition of ingredients which tend to form a network structure in the product, such as gums or fats, high solids levels such as high sugar levels, and low phase volumes for the ice may lead to a lower aspect ratio than in products without these ingredients or with higher ice phase volumes.

It has proved difficult to reproducibly produce a frozen food product having the desired texture and eating characteristics. Thus, there is a continuing need for methods for formulating frozen food products which on the one hand are less brittle and on the other hand retain improved ice-recrystallization and temperature tolerance properties upon longer term storage.

In one approach to extending storage life of frozen food, a variety of coatings to be applied to food have been developed. Exemplary of such coatings, U.S. Pat. No. 4,196,219 to Shaw, et al., issued Apr. 1, 1980, discloses a method of extending the storage life of cooked foods, such as meats, poultry, and fish, in the frozen state, comprising cooking the food, coating the cooked food with an edible coating the composition of which comprises the calcium salt of carrageenan, freezing the food coated with said calcium salt of carrageenan, and storing said coated food in the frozen state. It is disclosed that such coatings extend the storage life, reduce the rate of moisture loss, and create no objectionable flavors due to the coating.

Similarly, U.S. Pat. No. 6,200,622 to Darling, et al., issued Mar. 13, 2001, discloses a process for the production of a frozen food product comprising anti-freeze peptide (AFP), wherein the product is at least partially pre-frozen in the substantial absence of free AFP, followed by including the free AFP therein. Darling provides for frozen food products containing AFPs and having a non-brittle texture, said texture being maintained upon prolonged storage at low temperatures.

In addition, methods for the process of freezing food have been tried with food products of the type comprising a plurality of discrete pieces or portions. U.S. Pat. No. 6,007,859 to Taylor, et al., issued Dec. 28, 1999, discloses a method of coating a product with a liquid coating in a cooling chamber, comprising the steps of measuring the mass of the product either in the cooling chamber or just before it is introduced into the cooling chamber; the mass of a liquid cryogen which will suffice, when introduced into the cooling chamber and into contact with the product, to reduce the temperature of the mass of product to a first predetermined temperature below the melting point temperature of the coating material is calculated; the introduction of the calculated mass of liquid cryogen into the cooling chamber is controlled and the product is thereby cooled to the first predetermined temperature prior to application of a predetermined mass of coating material onto the cooled product to provide a coating thereon.

U.S. Pat. No. 6,284,298 to Montgomery, issued Sep. 4, 2001, discloses a method for preparing a frozen food product, comprising a variety of food pieces that have been precooked in the presence of water and subjected to a process in which substantially all of the free water present with the food pieces is removed; the food pieces are then passed through a continuous freezing unit, which increases the rigidity of the food pieces and leaves an amount of water on the surfaces of the food pieces; the food pieces are placed into a heat sealable container that includes a porous end piece along with measured amounts of a freezing gas and a seasoning, or adjuvant; the container is then sealed across the porous end piece and placed on a tumbling mechanism that coats the food pieces with the adjuvant and completes the freezing process as the freezing gas escapes through the porous end piece on the container.

Other approaches to extending the useful storage limits of frozen foods are known. For example, U.S. Pat. No. 6,020,013 to Kozma, issued Feb. 1, 2000, discloses a method of preventing freezer burn on frozen foods over an extended period of time. This is accomplished by providing a triple seal to prevent the ingress of ambient air through the seals into the interior of the storage bag which causes freezer burn. This is accomplished by providing an outer liquid impervious interlocking reclosable seal and a second liquid impervious interlocking reclosable inner seal adjacent and parallel to the outer seal. When food is placed in the storage bag, the inner seal is closed, water is put in the mouth of the storage bag above the sealed inner seal and below the open outer seal. The outer seal is then closed forming a triple seal closure that completely eliminates any possibility of ambient air entering into the interior of the storage bag.

Another approach to solving frozen storage problems is shown in U.S. Pat. No. RE37,892 to Kukal, et al., issued Oct. 22, 2002, which discloses a method for determining the optimum bio-storage temperature of biological materials. The temperature is between the melting point depression of the biological material and zero degrees C. The melting point depression temperature is determined by thermography, differential scanning calorimetry, and cryomicroscopy.

LEA Proteins. In plants, desiccation tolerant cells may have the ability to retain cellular moisture level or control the rate of water loss so as to maintain structural stability. Along with other desiccation-resistant proteins, late embryogenesis abundant proteins (hereinafter “LEA proteins”) are thought to control the hydration status of the plant cells in which they are found. For example, heat-soluble protein mixtures partially purified from wheat embryos dissolve quickly and dry slowly in the presence of sucrose, but have no unusual hydration characteristics in the absence of sugars. Prior to Applicants' work, is was believed that such proteins, in combination with sugars, functioned to control drying rate so that plant cells can better maintain critical water potentials.

Members of the LEA family of proteins were first characterized in cotton as a set of proteins that are highly accumulated in the embryos at the late stage of seed development. In general, LEA proteins have been proposed to bind to water, sequester ions, and protect proteins and membranes from desiccation in plants. Late embryogenesis abundant Group 1 (hereinafter “LEA-1”) proteins are involved in controlling the hydration status of plant cells. LEA-1 proteins were thought to interact with water and control water loss in cells exposed to desiccation. Prior to Applicants' work, most of the suggested roles for LEA-1 proteins were hypothetical and the exact protective mechanisms of LEA proteins were largely unknown.

LEA-1 proteins are distinguished from other groups of LEA proteins by being very hydrophilic, composed largely of random coils, and highly conserved along the entire length of the protein. LEA proteins also have a high proportion of glycine, glutamate, and glutamine residues. An LEA-1 protein is further characterized by having an internal 20 amino acid signature motif, repeated up to four times depending on the organism from which it is isolated. For example, these signature repeats are evident in a bacterial analogue protein, Bacillus subtilis GsiB stress protein, which is induced by glucose or phosphate starvation, oxygen limitation, heat, oxidation, and salinity.

U.S. Pat. No. 5,981,842 to Wu, et al., issued Nov. 9, 1999, discloses a method of producing a cereal plant cell or protoplast useful for regeneration of a drought stress or salt stress tolerant cereal plant by transforming the cereal plant cell or protoplast with a nucleic acid encoding a Group 1, 2, or 3 late embryogenesis abundant protein; specifically, the HVA1 gene from barley (Hordeum vulgare L.) was transformed into rice. A transgenic cereal plant or cereal plant cell or protoplast transformed with a nucleic acid encoding a late embryogenesis abundant protein is also disclosed.

Surprisingly, the present inventive subject matter provides a new absorbent material having desirable characteristics. LEA-1 proteins are uniquely applicable to a number of uses requiring water absorbency or water retention. In particular, such compositions are useful in absorbent materials and compositions, in topical therapeutics, as a pharmaceutical or cosmetic excipient, and as cryoprotectants for food and biologically relevant molecules. Further, the soybean GmD-19 protein in particular provides unexpectedly effective stress resistance in transgenic plants.

In this regard, it would be desirable to have absorbent materials and cryoprotectants formed from a natural product. It would be beneficial to have a non-toxic product derived from natural sources that would cause no concern when leachate from the material contacts the body or the material itself contacts the body or is ingested. It would be most desirable to have a hydrogel made of natural products and formable by adding water to a powder or fiber. Further, it would be desirable to have a biocompatible carrier or excipient that could be used in the topical delivery of active agents to the body. The inventive subject matter provides new compounds, compositions, and methods of use in response to these needs.

### SUMMARY OF THE INVENTIVE SUBJECT MATTER

The present inventive subject matter relates to a method for increasing the water absorbency or water retention capacity of a substrate material, said method comprising the steps of:

(i) combining an LEA-1 protein with said substrate material to form a mixture; and

(ii) forming said mixture into a configuration in which said LEA-1 protein contacts water molecules upon exposure to an environment comprising said water molecules.

In another embodiment, the inventive subject matter further relates to an absorbent product produced by the process of:

(i) combining a first component comprising an LEA-1 protein with a composition to form a mixture; and

(ii) forming said mixture into a configuration in which said LEA-1 protein contacts water molecules upon exposure to an environment comprising said water molecules.

In another embodiment, the inventive subject matter further relates to a pharmaceutical composition comprising:

(i) an active therapeutic agent;

(ii) an LEA-1 protein; and

(iii) a pharmaceutically acceptable carrier.

In another embodiment, the inventive subject matter further relates to a drug delivery system comprising an LEA-1 protein and an active agent.

In another embodiment, the inventive subject matter further relates to a method for maintaining or increasing the hydration state of a pharmaceutical or cosmetic composition, said method comprising combining an LEA-1 protein with said pharmaceutical or cosmetic composition.

In another embodiment, the inventive subject matter further relates to a method for improving the palatability of frozen food, comprising treating said food with an LEA-1 protein.

In another embodiment, the inventive subject matter further relates to a method for improving the storage life of frozen food, comprising treating said food with an LEA-1 protein.

In another embodiment, the inventive subject matter further relates to a method for maintaining the integrity of a biological structure upon freezing, comprising treating said biological structure with an LEA-1 protein.

In another embodiment, the inventive subject matter further relates to a method for increasing the resistence of an organism to drought stress, osmotic stress, heat stress, freezing stress, or a combination thereof, comprising transfecting said organism with an expression vector comprising an isolated GmD-19 DNA sequence operably linked to a promoter which is constitutively or inducibly expressed in said organism.

### BRIEF DESCRIPTION OF THE DRAWINGS

FIG. 1(A) is a photograph of an SDS-PAGE gel which depicts expression and purification of group 1 LEA protein from E. coli at different times following IPTG induction.

FIG. 1(B) is a photograph of an SDS-PAGE gel which depicts composite purification steps of rGmD-19 treated with boiling.

FIG. 1(C) is a photograph of an SDS-PAGE gel which depicts composite purification steps of rGmD-19 without boiling.

FIG. 2(A) is a graph which depicts a heating thermogram of BSA, with and without boiling, using differential scanning colorimetry.

FIG. 2(B) is a graph which depicts a heating thermogram of rGmD-19, with and without boiling, using differential scanning colorimetry.

FIG. 3 is a graph which depicts UV-absorption spectrum of rGmD-19. The inset graph depicts the expanded near-UV region of the spectrum on a smaller absorbance scale.

FIG. 4(A) is a graph which depicts the effect of temperature on the second derivative spectrum of rGmD-19.

FIG. 4(B) is a graph which depicts the effect of temperature on the difference between the second derivative values at 279 nm and 283 nm.

FIG. 5(A) is a graph which depicts water absorption isotherms of various proteins over the full relative humidity range.

FIG. 5(B) is a graph which depicts water absorption isotherms of various proteins at low relative humidity.

FIG. 6(A) is a graph which depicts a differential scanning calorimetric scan at different moisture levels for rGmD-19.

FIG. 6(B) is a graph which depicts a differential scanning calorimetric scan at different moisture levels for rGmDhn.

FIG. 6(C) is a graph which depicts a differential scanning calorimetric scan at different moisture levels for BSA.

FIG. 6(D) is a graph which depicts a differential scanning calorimetric scan at different moisture levels for Gluten.

FIG. 7(A) is a graph which depicts unfrozen water content of rGmD-19.

FIG. 7(B) is a graph which depicts unfrozen water content of rGmDhn.

FIG. 7(C) is a graph which depicts unfrozen water content of BSA.

FIG. 7(D) is a graph which depicts unfrozen water content of Gluten.

FIG. 8 is a drawing which depicts the T-DNA vector expressing GmD-19, as described in the Examples herein.

FIG. 9(A) is a photograph which depicts a Northern blot of GmD-19 transgene expression.

FIG. 9(B) is a photograph which depicts a Western blot of GmD-19 transgene expression.

FIG. 10(A) is a bar graph which depicts germination rate of GmD-19 transgenic Arabidopsis grown in NaCl solution.

FIG. 10(B) is a bar graph which depicts survival rate of GmD-19 transgenic Arabidopsis grown in NaCl solution.

FIG. 10(C) is a bar graph which depicts fresh weight of GmD-19 transgenic Arabidopsis grown in NaCl solution.

FIG. 10(D) is a bar graph which depicts dry weight of GmD-19 transgenic Arabidopsis grown in NaCl solution.

FIG. 11(A) is a bar graph which depicts germination rate of GmD-19 transgenic Arabidopsis grown in sorbitol solution.

FIG. 11(B) is a bar graph which depicts root length of GmD-19 transgenic Arabidopsis grown in sorbitol solution.

### DETAILED DESCRIPTION OF THE INVENTIVE SUBJECT MATTER

### Definitions

The term “LEA-1 protein” as used herein refers to a late embryogenesis abundant Group 1 protein, as further described herein.

The term “water absorbency” as used herein refers to the capacity of a material to absorb water from external surroundings.

The term “water retention capacity” as used herein refers to the capacity of a material to resist the loss of water to external surroundings.

The term “substantially water-insoluble bond” as used herein refers to a chemical bond, whether a covalent bond, an ionic bond, or a hydrogen bond, which is sufficiently strong to survive intact upon exposure to water of the material(s) bonded.

The term “co-express” as used herein refers to two or more gene products which are produced at the same time.

The terms “transfecting” and “transfection” as used herein refer to the process of producing genetic alteration of a cell or organism using recombinant DNA technology.

The term “vector” as used herein refers to the DNA of any transmissible agent into which a segment of foreign DNA can be cloned, in order to introduce and express such foreign DNA in a target host cell. Vectors include, for example, plasmid vectors, viral vectors, bacteriophage vectors, cosmid vectors, phagemid vectors, and phasmid vectors, which are selected according to the target cell type.

The term “expression vector” as used herein refers to a vector which carries a promoter and promotes transcription of a cloned DNA segment.

The term “promoter” as used herein refers to a DNA sequence which indicates a transcription initiation site and is generally located upstream of the 5′ end of a gene.

The terms “palatable” or “palatability” as used herein refers to a subjective judgment that a foodstuff is acceptable to the taste or sufficiently agreeable in flavor to be consumed.

The term “resistence” as used herein refers to the ability of a living organism to resist the effects of a disadvantageous environment or substance.

The term “active therapeutic agent” as used herein refers to any substance or substances comprising a drug, active therapeutic substance, metabolite, medicament, vitamin, or mineral; any substance used for treatment, prevention, diagnosis, cure, or mitigation of disease or illness; any substance which affects anatomical structure or physiological function; or any substance which alters the impact of external influences on an animal, or metabolite thereof, and as used herein encompasses the terms “active substance”, “therapeutic substance”, “agent”, “active agent”, “drug”, “medication”, “medicine”, “medicament”, “biologically active substance”, and other such similar terms.

Methods Relating to Hydration State and Water Absorbency

The soybean, Glycine max group 1 late embryogenesis abundant gene, GmD-19, encodes an 11.5 kDa, glycine-rich protein. Like other group 1 LEA proteins, GmD-19 has a preponderance of polar residues such as Gly, Gln, lacks non-polar residues such as Cys, Trp, and contains a high proportion of charged residues, such as Asp, Glu. Group 1 LEA proteins in plants are further distinguished by an internal 20-amino-acid sequence motif tandemly repeated from one to four times depending on the individual protein.

The encoded protein from Glycine max was overexpressed in E. coli and isolated to homogeneity. UV spectroscopy and far-UV CD analysis of the purified protein indicate that it forms about 82.5% random coil structures and contains solvent-interacting left-handed extended helical or poly (L-proline)-type, PII, type structures. As demonstrated by GmD-19, LEA-1 proteins have several unique properties:

1) LEA-1 proteins retain water at very low relative humidity compared to other LEA proteins, so that its water content remains high under extremely dry conditions;

2) LEA-1 proteins exhibit a high water retention capacity compared to other LEA proteins under extremely dry conditions; and

3) LEA-1 proteins absorb large amounts of water under high relative humidity and exhibits the highest hydration level in relation to other proteins over a large range of relative humidity.

Applicants produced and purified an LEA-1 protein, GmD-19, which was then tested for its physiochemical properties, including its capacity to hold and absorb water, and to affect the thermal behavior of aqueous solutions. The protein retains or absorbs at least 3 times more water than other control water-soluble proteins such as bovine serum albumin. Without being bound by any particular theory, it is expected that these results indicate that LEA-1 protein interacts strongly with water, resulting in an unusual ability to alter hydration properties so that water contents remain high but molecular motions are somewhat restricted in the aqueous environment. Differential scanning calorimetry measurements document that GmD-19 affects the thermal behavior of aqueous solutions: the much higher unfrozen water content, which was nearly twice that of control proteins such as BSA and gluten, suggests that molecular motions of water molecules are sufficiently restricted to limit phase transitions. DSC scans of GmD-19 showed several first and second order transitions and unusually low melting temperatures at relatively high water contents, indicating altered phase behavior and the occurrence of amorphous and polymorphic crystalline structures of LEA-1/water solutions. This quality supports the use of LEA-1 proteins for retaining moisture over a wide range of relative humidities.

Thus, the present inventive subject matter relates to a method for increasing the water absorbency or water retention capacity of a substrate material, said method comprising the steps of:

(i) combining an LEA-1 protein with said substrate material to form a mixture; and

(ii) forming said mixture into a configuration in which said LEA-1 protein contacts water molecules upon exposure to an environment comprising said water molecules.

In an aspect of the inventive subject matter, said water molecules are selected from the group consisting of water vapor, liquid water, or ice. However, the inventive subject is not so limited, as it is expected that conceivably an application for the inventive subject matter in an environment of supercritical water exists or will exist, which would not fall squarely within any of the recited three states of matter for water.

In another aspect of the inventive subject matter, said substrate material is selected from the group consisting of paper products, cloth or fabric, hydrogel, foam, resin, and polymer matrix.

In another aspect of the inventive subject matter, said method comprises the additional step of binding said mixture together to form substantially water-insoluble bonds between said LEA-1 protein and said substrate material.

LEA-1 protein also is useful as a moisturizing agent in pharmaceutical and cosmetic formulations. Its ability to bind and retain water make it a useful component in moisturizing creams, lotions, soaps, shampoos, wound dressings, and artificial skin. Under drying conditions, LEA-1 protein maintains a high level of hydration, and has the desirable capacity to keep mixtures fluid. Under extremely moist conditions, LEA-1 protein is expected to have a relative drying effect, adsorbing free water and sequestering water from a local environment.

Skin moisturizers are intended to increase water content of the outer layers of the skin. Moisture retention by current skin moisturizers is transient, as evaporation of emulsion water occurs rapidly, often within seconds or minutes, reducing the effectiveness of such products. Reduction of water loss would improve with formulations incorporating LEA-1 protein.

Epidermal absorption of lipid components of topical preparations are understood to be longer lived events; however, oil-based components of such products can contribute to undesirable characteristics and effects of formulations incorporating them. Many formulations rely on glycerol as a humectant; however, glycerol has only a short-term effect on increasing skin moisture levels. It is expected that the quality and effectiveness of moisturizing agents would be dramatically improved with the addition of LEA-1 protein, which binds water even at very low humidity and then releases this bound water to surrounding tissues gradually.

Additionally, moisture-retentive wound dressings promote the healing process. Wound healing occurs at a greater rate when wound sites are kept moist, compared to conventional dressings. At the same time, the dressing must absorb wound exudates. A variety of films, hydrocolloidal fiber, gelatin, and polyethylene oxide gel wound dressing systems would be enhanced by the incorporation of an LEA-1 protein, which is expected to provide a high capacity to maintain the hydration state of the wound, while allowing drainage and oxygen exchange. An improvement in wound dressings, comprising the addition of an LEA-1 protein additive to bandages, is expected to provide the capacity to maintain constant moisture levels despite variations in wound exudate levels, represents a significant improvement in wound dressing technology.

Similarly, combining artificially grown skin with LEA-1 protein would be beneficial, as the LEA-1 protein would keep the artificial skin from drying out, which limits the effectiveness of grafts of the artificial skin. Examples of ways to protect artificial skin include, but are not limited to, spraying LEA-1 protein on the product before packaging and spraying or painting a solution of LEA-1 protein onto the in situ graft.

Thus, the present inventive subject matter further relates to a method for maintaining or increasing the hydration state of a pharmaceutical or cosmetic composition, said method comprising combining an LEA-1 protein with said pharmaceutical or cosmetic composition.

In an aspect of the inventive subject matter, said composition is a topical formulation.

In a preferred embodiment, said topical formulation is a transdermal drug delivery formulation.

In another aspect of the inventive subject matter, said composition is a sustained or controlled drug release system.

In another aspect of the inventive subject matter, said composition is a pharmaceutical or cosmetic gel, cream, lotion, ointment, soap, shampoo, solution, spray, jelly, gel, emulsion, wound dressing, suppository, or artificial skin membrane.

Genetic manipulation techniques may be used to produce LEA-1 proteins, as follows: An appropriate host cell or organism is transformed by a gene construct that contains the desired polypeptide. The nucleotide sequence coding for the polypeptide can be inserted into a suitable expression vector encoding the necessary elements for transcription and translation, and in such a manner that they will be expressed under appropriate conditions, for example in proper orientation and correct reading frame, and with appropriate targeting and expression sequences. The methods required to construct expression vectors are well known to those skilled in the art.

A number of expression systems may be utilized to express the polypeptide coding sequence for an LEA-1 protein. These include, but are not limited to, bacteria, yeast, insect cell systems, plant cell culture systems, and plants, each of which is transformed with an appropriate expression vector. A wide variety of plants and plant cell systems can be transformed with the nucleic acid constructs of the desired polypeptides.

### Processes for Making an Absorbent Product

For example, an LEA-1 protein is formed into a thin layer, with or without binders and other chemicals, and is then fabricated between layers of fine mesh or weave, which serve to contain the protein. However, other methods for containing the LEA protein in such absorbent products are envisioned, including binding said protein directly to a structural matrix material, in a manner that the LEA protein remains bound to the substrate upon exposure to water. In addition, LEA-1 protein is heat stable, resulting in greater ease of manufacture with heat-pressure processes.

Thus, the present inventive subject matter relates to an absorbent product produced by the process of:

(i) combining a first component comprising an LEA-1 protein with a composition to form a mixture; and

(ii) forming said mixture into a configuration in which said LEA-1 protein contacts water molecules upon exposure to an environment comprising said water molecules.

AS discussed above, in an aspect of the inventive subject matter, said water molecules are selected from the group consisting of water vapor, liquid water, or ice. However, the inventive subject is not so limited, as it is expected that conceivably an application for the inventive subject matter in an environment of supercritical water exists or will exist, which would not fall squarely within any of the recited three states of matter for water.

In another aspect of the inventive subject matter, said substrate material is selected from the group consisting of paper products, cloth or fabric, hydrogel, foam, resin, and polymer matrix.

In another aspect of the inventive subject matter, said product is selected from the group consisting of wound dressings, disposable diapers, adult incontinence pads, adult incontinence briefs, menstrual pads, sanitary napkins, and tampons.

### Pharmaceutical Compositions

In another embodiment, the inventive subject matter further relates to a pharmaceutical composition comprising:

(i) an active therapeutic agent;

(ii) an LEA-1 protein; and

(iii) a pharmaceutically acceptable carrier.

Without being limited thereto, said pharmaceutical composition optionally incorporates other excipients, including carriers, diluents, solvents, surface active agents, also called surfactants, binders and adhesives, lubricants, bulking substances, plasticizers, disintegrants, inert substrate particles, matrix forming excipients, colorants, sweeteners, flavoring agents, and miscellaneous materials such as buffers and adsorbents in order to prepare a particular medicated composition.

In a preferred embodiment, said carriers are selected from the group consisting of sugar, lactose, gelatin, starch, silicon dioxide, and mixtures thereof.

Exemplary non-limiting diluents which are of use according to the present inventive subject matter may be selected from the group consisting of calcium phosphate, calcium sulfate, carboxymethylcellulose calcium, cellulose, cellulose acetate, dextrates, dextrin, dextrose, fructose, glyceryl palmitostearate, hydrogenated vegetable oil, kaolin, lactitol, lactose, magnesium carbonate, magnesium oxide, maltitol, maltodextrin, maltose, microcrystalline cellulose, polymethacrylates, powdered cellulose, pregelatinized starch, silicified microcrystalline cellulose, sodium chloride, sorbitol, starch, sucrose, sugar, talc, hydrogenated vegetable oil, and mixtures thereof.

Examples of solvents useful in the present inventive compositions can be, but are not limited to, those selected from the group consisting of water, a volatile propellant, a C₁-C₆ fluid alkyl or branched alkyl alcohol, an aromatic alcohol, an ether of a sorbitol derivative, propylene carbonate, xylene, methylene chloride, ethylhexanediol, polysiloxanes, dimethyl ether, and mixtures thereof.

Preferred volatile propellants useful as solvents in the present inventive compositions include, but are not limited to, hydrocarbon propellants such as propane, isopropane, n-butane, and isobutene, chlorofluorocarbons (CFCs), hydrofluoroalkanes (HFAs), and dimethyl ether.

A wide variety of surfactants can be employed in the inventive compositions. These surfactants can include, for example, polyoxyethylene fatty ethers, polyoxyethylene fatty esters, fatty acids, sulfated fatty acids, phosphated fatty acids, sulfosuccinates, amphoteric surfactants, non-ionic poloxamers, non-ionic meroxapols, petroleum derivatives, aliphatic amines, polysiloxane derivatives, sorbitan fatty acid esters, pharmaceutically acceptable salts thereof, and mixtures thereof.

Other surfactants commonly known as useful in the preparation of foamable compositions are further contemplated as within the scope of the present inventive subject matter. These other surfactants include, for example, those listed in the CTFA Cosmetic Ingredient Dictionary, Second Edition, The Cosmetic Toiletry and Fragrance Association, Inc., 1133 Fifteenth Street, N.W., Washington, D.C. 20005, 1977, the entire contents of which are hereby incorporated by reference.

A binder is generally present in amounts of 1.0 to 10.0% by weight of an encapsulated product. Binders suitable for use in the present inventive subject matter include, without limitation, plasdone, povidone, pharmaceutical glaze, sugar, hydroxypropylmethylcellulose, hydroxyethylcellulose, hydroxypropylcellulose, acrylic and methacrylic acid co-polymers, carboxymethylcellulose, ethylcellulose, methylcellulose, sodium alginate, alginic acid, acacia, dextrin, gelatin, liquid glucose, hydrogenated vegetable oil, magnesium aluminum silicate, maltodextrin, polyethylene oxide, polymethacrylates, starch, zein, gums such as guar gum, and milk derivatives such as whey and starches, other conventional binders well known to persons skilled in the art, and mixtures thereof.

Exemplary non-limiting lubricants which are of use as other excipients according to the present inventive subject matter may be selected from the group consisting of calcium stearate, canola oil, glyceryl palmitostearate, hydrogenated vegetable oil, magnesium oxide, mineral oil, poloxamer, polyethylene glycol, polyvinyl alcohol, sodium benzoate, sodium lauryl sulfate, sodium stearyl fumarate, stearic acid, sterilizable corn starch, talc, zinc stearate, and mixtures thereof.

Exemplary non-limiting bulking substances which are of use as other excipients according to the present inventive subject matter may be selected from the group consisting of sugar, lactose, gelatin, starch, silicon dioxide, and mixtures thereof.

Exemplary non-limiting plasticizers are selected from the group consisting of lanolin, mineral oil, petrolatum, benzyl phenylformate, chlorobutanol, glycerol, triacetin, diethyl phthalate, diethyl sebacate, triethyl citrate, crotonic acid, propylene glycol, castor oil, citric acid esters, butyl phthalate, dibutyl phthalate, dibutyl sebacate, polyethylene glycols, benzyl benzoate, glycerin, sorbitol, tributyl citrate, acetyltriethyl citrate, glyceryl triacetate, glyceryl tributarate, glyceryl diacetate, acetylated monoglycerides, other excipients, and mixtures thereof. As is evident, plasticizers may be hydrophobic as well as hydrophilic in nature.

Exemplary non-limiting disintegrants which are of use as other excipients according to the present inventive subject matter may be selected from the group consisting of alginic acid, carboxymethylcellulose, hydroxypropyl cellulose, microcrystalline cellulose, colloidal silicon dioxide, croscarmellose sodium, crospovidone, magnesium aluminum silicate, methylcellulose, polacrilin, povidone, sodium alginate, sodium starch glycolate, starch, and mixtures thereof.

Non-limiting examples of inert substrate particles are sugar spheres and non-toxic plastic resin beads.

Exemplary non-limiting colorants which are of use as other excipients according to the present inventive subject matter may be selected from the group consisting of curcumin, lactoflavin (riboflavin), tartrazine, quinoline yellow, sunset yellow FCF, cochineal carminic acid, carmoisine, ponceau 4R, patent blue V, indigo carmine, chlorophylls, lissamine green, caramel, black PN, carbo medicinalis vegetabilis, carotenoids, xanthophylls, betanin, anthocyanins, calcium carbonate, titanium dioxide, iron oxides and hydroxides, indigotine, alphazurine FG, indanthrene blue, fast green FCF, alizarin cyanine, quinizarine green SS, pyranine concentrated, orange II, dibromofluorescein, diiodofluorescein, erythrosine, ponceau SX, lithol rubin B, toney red, tetrabromofluorescein, eosine, tetrachlorotetrabromofluorescein, phloxine B, helindone pink CN, brilliant lake red R, acid fuchsine, lake bordeaux B, flaming red, alba red, allura red AC, alizurol purple SS, tartrazine, sunset yellow FCF, fluorescein, naphthol yellow S, uranine, quinoline yellow, alumina, aluminum powder, annatto extract, beta-carotene, bismuth oxychloride, bronze powder, calcium carbonate, canthaxanthin, chromium-cobalt-aluminum oxide, chromium hydroxide green, cochineal extract, copper powder, dihydroxy acetone, ferric ammonium citrate, ferric ammonium ferrocyanide, ferric ferrocyanide, guanine, iron oxides synthetic, logwood extract, mica, potassium sodium copper chlorophyllin, pyrogallol, pyrophyllite, talc, zinc oxide, and mixtures thereof.

Exemplary non-limiting sweeteners which are of use as other excipients according to the present inventive subject matter may be selected from the group consisting of acesulfame potassium, aspartame, dextrose, fructose, liquid glucose, glycerol, lactitol, lactose, maltitol, maltose, saccharin, saccharin sodium, sodium cyclamate, sorbitol, sucrose, confectioner's sugar, xylitol, and mixtures thereof.

Exemplary non-limiting flavoring agents which may be used include those flavors known to the skilled artisan, such as natural and artificial flavors. These flavorings may be chosen from synthetic flavor oils and flavoring aromatics and/or oils, oleoresins and extracts derived from plants, leaves, flowers, fruits, and so forth, and combinations thereof. Non-limiting representative flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Also useful flavorings are artificial, natural and synthetic fruit flavors such as vanilla, and citrus oils including, without limitation, lemon, orange, lime, grapefruit, and fruit essences including apple, pear, peach, grape, strawberry, raspberry, cherry, plum, pineapple, apricot and so forth. These flavoring agents may be used in liquid or solid form and may be used individually or in admixture. Commonly used flavors include mints such as peppermint, menthol, artificial vanilla, cinnamon derivatives, and various fruit flavors, whether employed individually or in admixture.

Other useful flavorings include aldehydes and esters such as cinnamyl acetate, cinnamaldehyde, citral diethylacetal, dihydrocarvyl acetate, eugenyl formate, p-methylamisol, and so forth may be used.

If the flavor to be added is liquid, then the liquid flavor is first absorbed onto a solid absorbent. Examples of absorbents on which the liquid may be absorbed include, without limitation, silica gel particles, starches, carbohydrates such as sugars and polyhydroxyalcohols, celluloses, calcium salts such as calcium phosphate, calcium carbonate, and calcium sulfonate, and other absorbing agents in free-flowing powder form. The amount of liquid flavor added depends on the final concentration desired. Generally, though, the liquid flavor will be present in quantities from about 0.1% to 70% by weight of the resultant flavor/absorbent mixture.

In addition to the active therapeutic agent and the excipients listed above, the pharmaceutical preparations of the invention may additionally comprise other nonessential, optional ingredients known to a person of ordinary skill in the art as suitable for a pharmaceutical composition. For example, a topical preparation may optionally further include one or more preservatives well known in the art, such as benzoic acid, sorbic acid, methylparaben, propylparaben, ethylenediaminetetraacetic acid (EDTA), benzyl alcohol, phenoxyethanol, DMDM hydantoin, and imidazolidinyl urea. These preservatives may be present in amounts up to about 1% and preferably from about 0.05 to about 0.5% by weight of the pharmaceutical composition. Other additional optional ingredients may include thickeners and viscosity modifiers such as diethanolamide of a long chain fatty acid, fatty alcohols (i.e. cetearyl alcohol), sodium chloride, sodium sulfate, ethyl alcohol, hydroxyethyl cellulose, and Carbomer; coloring agents such as any of the FD&C or D&C dyes; hair oxidizing (bleaching) agents such as hydrogen peroxide, perborate salts, and persulfate salts; hair reducing agents such as the thioglycolates; perfumes; moisturizers, emollients; plasticizers; stabilizers; skin penetrating agents; and chelating agents such as disodium EDTA.

Additionally, the present inventive pharmaceutical compositions optionally contain a moisturizing agent. Non-limiting examples of such moisturizing agents include C₃-C₆ diols and triols, glycerin, sorbitol, propylene glycol, dipropylene glycol, 1,3-butylene glycol, glucose, xylitol, maltitol, polyethylene glycol, hyaluronic acid, chondroitin sulfuric acid, polyoxyethylene methylglycoside, pyrrolidone carboxylate salts, and polyoxypropylene methylglycoside. This moisturizing agent acts to further enhance the effects and curative action on the skin. Further, the moisturizing agent moisturizes the skin, avoiding effects such as drying, redness, blistering, burning, itching, and peeling of the skin. Accordingly, the addition of the moisturizing agent to the present compositions may improve patient compliance with a prescribed treatment regimen.

The present inventive compositions are optionally formed as an oil-in-water emulsion, i.e. an emulsion having an oil phase and an aqueous phase. Preferably, the oil phase of the emulsion comprises an oily material and an emulsifier to aid in formation of the emulsion. More preferably, the oil phase contains at least two emulsifiers.

Non-limiting exemplary oily materials include mineral oil, petrolatum, petroleum derivatives, fatty acids, fatty acid derivatives, fatty alcohols, fatty alcohol derivatives, paraffins, and mixtures thereof.

At least one emulsifier is used to form an emulsion. In a preferred embodiment, at least two emulsifiers are present in an oil phase to help form the emulsion. Preferred, non-limiting examples of emulsifiers used in the present inventive compositions include polyoxyethylene sorbitan fatty acid esters, sorbitan fatty acid esters, propylene glycol stearate, glyceryl monostearate, polyethylene glycol, fatty alcohols, polymeric ethylene oxide-propylene oxide block polymers, derivatives thereof, pharmaceutically acceptable salts thereof, and mixtures thereof. In a preferred embodiment, the emulsifiers used in the present inventive compositions are either naturally or synthetically prepared.

The present inventive compositions may further comprise several additional excipients commonly known to those of ordinary skill in the art as useful in topical compositions. Several non-limiting examples of such additional excipients include antioxidants, chelates, preservatives, emollients, humectants, fluid alkyl alcohols, thickening agents, pH modifier, and mixtures thereof.

Non-limiting examples of specific antioxidants useful in the present inventive compositions include ascorbic acid, fumaric acid, malic acid, alpha tocopherol, ascorbic acid palmitate, butylated hydroxyanisole, propyl gallate, sodium ascorbate, sodium metabisulfite, and mixtures thereof.

Non-limiting examples of specific preservatives useful in the present inventive compositions include methylparaben, benzalkonium chloride, propylparaben, benzoic acid, EDTA, phenolic acid, sorbic acid, benzyl alcohol, isopropyl alcohol, benzethonium chloride, bronopol, butylparaben, cetrimide, chlorhexidine, chlorobutanol, chlorocresol, cresol, ethylparaben, glycerol, imidurea, phenol, phenoxyethanol, phenylethyl alcohol, potassium sorbate, propylene glycol, sodium benzoate, sodium propionate, sorbic acid, thimerosol, and mixtures thereof.

Non-limiting examples of specific emollients useful in the present inventive compositions include myristyl lactate, isopropyl palmitate, light liquid paraffin, cetearyl alcohol, lanolin, mineral oil, petrolatum, ceryl esters wax, cholesterol, glycerol, glycerol monostearate, isopropyl myristate, lecithin, and mixtures thereof.

Non-limiting examples of specific humectants useful in the present inventive compositions include glycerin, propylene glycol, sorbitol, and triacetin.

Non-limiting examples of specific fluid alkyl alcohols useful in the present inventive compositions include ethanol, isopropyl alcohol, octodecyl alcohol, propyl alcohol, butanol, and pentanol.

Non-limiting examples of specific thickening agents useful in the present inventive compositions include cetyl alcohol, Carbomers, acrylates/C10-30 alkyl acrylate crosspolymers, hydroxyethylcellulose, hydroxypropylcellulose, polyethylene oxide, and mixtures thereof.

The pH modifiers useful in the present inventive compositions include acids, bases, and mixtures thereof. Preferred non-limiting examples of pH modifiers in this regard include acetic acid, acetylsalicyclic acid, ascorbic acid, boric acid, carbonic acid, citric acid, formic acid, ethanesulfonic acid, fumaric acid, glycerophosphoric acid, hippuric acid, hydrochloric acid, maleic acid, methanesulfonic acid, nitrous acid, oxalic acid, phosphoric acid, saccharin, sorbic acid, sulfuric acid, thiosulfuric acid, undecylenic acid, ethanolamine, triethanolamine, sodium carbonate, sodium acetate, sodium hydrogen phosphate, sodium dihydrogen phosphate, sodium citrate, sodium bicarbonate, sodium hydroxide, and mixtures thereof.

The present inventive pharmaceutical compositions may also be packaged in a container suitable for storage and delivery of said composition.

The present pharmaceutical compositions are particularly useful in topical formulations for treating a wide variety of dermatological diseases, disorders, or conditions in a mammal, especially diseases affecting mammalian tissue. Examples of such diseases include, but are not limited to, eczema, infantile eczema, psoriasis, scalp psoriasis, atopic dermatitis, dermatitis herpetiformis, contact dermatitis, seborrheic dermatitis, neurodermatitis, pruritis, fungal diseases, and intertrigo.

It is contemplated that the inventive pharmaceutical compositions may optionally incorporate any active therapeutic agent which is compatible with water. A non-limiting list of such materials includes the following: antitussives, antihistamines, decongestants, alkaloids, mineral supplements, laxatives, vitamins, antacids, ion exchange resins, anti-cholesterolemics, antiarrhythmics, antipyretics, analgesics, appetite suppressants, expectorants, anti-anxiety agents, anti-ulcer agents, anti-inflammatory substances, coronary dilators, cerebral dilators, peripheral vasodilators, anti-infectives, psycho-tropics, antimanics, stimulants, gastrointestinal agents, sedatives, antidrrheal preparations, anti-anginal drugs, vasodialators, anti-hypertensive drugs, vasoconstrictors, migraine treatments, antibiotics, tranquilizers, anti-psychotics, antitumor drugs, anticoagulants, antithrombotic drugs, hypontics, anti-emetics, anti-nausants, anti-convulsants, neuromuscular drugs, hyper- and hypoglycemic spasmodics, uterine relaxants, mineral and nutritional additives, antiobesity drugs, anabolic drugs, erythropoetic drugs, antiashmatics, cough suppressants, mucolytics, anti-uricemic drugs and mixtures thereof.

Examples of preferred active therapeutic agents useful in the present inventive compositions can be, but are not limited to, those selected from the group consisting of steroids, antifungal agents, antimicrobials, agents intended to protect the skin, modify its appearance, or improve its rate of healing, and mixtures thereof.

In a preferred embodiment, said steroid is a corticosteroid that works to beneficially alter the appearance, metabolic or functional state, permeability, or health of a living organism. Corticosteroids are steroid hormones produced by the cortex of the adrenal gland. Preferred corticosteroids useful in the present inventive compositions include, but are not limited to, alclometasone dipropionate, amcinonide, beclamethasone dipropionate, betamethasone benzoate, betamethasone dipropionate, betamethasone valerate, budesonide, clobetasol propionate, clobetasone butyrate, cortisone acetate, desonide, desoximetasone, diflorasone diacetate, diflucortolone valerate, fluclorolone acetonide, flumethasone pivalate, fluocinolone acetonide, fluocinonide, fluocortin butyl, fluocortolone preparations, fluprednidene acetate, flurandrenolide, flurandrenolone, fluticasone propionate, halcinonide, halobetasol propionate, hydrocortisone, hydrocortisone acetate, hydrocortisone butyrate, hydrocortisone propionate, hydrocortisone valerate, methylprednisolone acetate, mometasone furoate, pramoxine hydrochloride, prednisone acetate, prednisone valerate, triamcinolone acetonide, and mixtures thereof.

In another preferred embodiment, said active therapeutic agent is an antifungal agent which can include, but is not limited to, those selected from the group consisting of imidazoles, hydroxy pyridones, triazoles, allyl amines, undecylenic acid derivatives, tolnaftate, haloprogin, pyridinethiones, cloquinol, and mixtures thereof.

Preferred antifungal agents useful in the present inventive compositions include, but are not limited to, amphotericin B, butoconazole nitrate, ciclopirox olamine, clindamycin, clioquinol, clotrimazole, econazole, econazole nitrate, fluconazole, flucytosine, griseofulvin, itraconazole, ketoconazole, miconazole, micronazole, naftifine, nystatin, omadine disulfide, sulconazole, terbinafine, terconazole, tioconazole, tolnaftate, triacetin, unecylenic acid, zinc pyrithione, and mixtures thereof.

In another preferred embodiment, said active therapeutic agent is an antimicrobial agent which can include, but is not limited to, those selected from the group consisting of amikacin, bacitracin, colistin, gentamicin, kanamycin, metronidazole, mupirocin, neomycin, netilmicin, polymyxin B, streptomycin, tobramycin, phenols and cresols such as 2,4-dichloro-sym-metaxylenol, parachlorometaxylenol, and parachlorometacresol, bisphenols such as hexachlorophene, dichlorophene, bithionol, triclosan, and fentichlor, salicylanilides such as 4′,5-dibromsalicylanilide, 3′,4′,5-trichlorosalicylanilide, 3′,4′,5-tribromosalicylanilide, and 3,5,dibromo-3′-trifluoromethyl-salicylanilide, carbanilides such as trichlorocarbanilde and 3-trifluoromethyl-4-4′-dichlorocarbanilide, quaternary ammonium compounds such as alkyl-dimethyl benzyl ammonium chloride, alkyl-trimethyl ammonium chloride, alkyl trimethyl ammonium bromide, cetyl-trimethyl ammonium bromide, B-phenoxyethyl-dimethyl-dodecyl ammonium bromide, p-tert-octylphenoxyethoxyethyl-dimethyl-benzyl ammonium chloride, tetradecyl-pyridinium bromide, cetyl pyridinium bromide, cetyl pyridinium chloride, di-(n-octyl)-dimethyl ammonium bromide, alkyl-isoquinolinium bromide, 1-(3-chloroallyl)-3-5-7-triaza-1-azoniaadamantane chloride, and chlorhexidine (1,6,di(N-p-chlorophenylguanidino)hexane), 2-bromo-2-nitropropan-1,3-diol, imidazonidyl urea, ethanol, isopropyl alcohol, and mixtures thereof.

In another preferred embodiment, said active therapeutic agent is a skin-conditioning agent. Preferably, the skin-conditioning agent is selected from the group consisting of hydrocarbon oils and waxes, silicones, fatty acid derivatives, cholesterol, cholesterol derivatives, di- and tri-glycerides, vegetable oils, vegetable oil derivatives, liquid nondigestible oils such as those described in Mattson, U.S. Pat. No. 3,600,186, and Jandacek et al., U.S. Pat. Nos. 4,005,195 and 4,005,196, all of which are herein incorporated by reference in their entirety, or blends of liquid digestible or nondigestible oils with solid polyol polyesters such as those described in Jandacek, U.S. Pat. No. 4,797,300, and Letton, U.S. Pat. Nos. 5,306,514, 5,306,516, and 5,306,515, all of which are herein incorporated by reference in their entirety, acetoglyceride esters, alkyl esters, alkenyl esters, lanolin and its derivatives, milk tri-glycerides, wax esters, beeswax derivatives, sterols, phospholipids, and mixtures thereof.

In another preferred embodiment, said active therapeutic agent is an UV absorber/sunscreen agent. Preferably, the UV absorber/sunscreen agent is selected from the group consisting of para-aminobenzoic acid and its derivatives (ethyl, isobutyl, glyceryl esters), p-dimethylaminobenzoic acid and its derivatives (ethyl, isobutyl, glyceryl esters), o-aminobenzoates and its derivatives (methyl, menthyl, phenyl, benzyl, phenylethyl, linaly, terpenyl, and cyclohexenyl esters), salicylates (amyl, phenyl, benzyl, menthyl, glyceryl, and dipropylene-glycol esters), cinnamic acid derivatives (menthyl and benzyl esters; alphphenyl cinnamonitrile; butly cinnamoyl pyruvate, 2-ethylhexyl p-methoxycinnamate, iso-amyl p-methoxycinnamate), dihydroxycinnamic acid derivatives (umbelliferone, methyl-umbelliferone, methylaceto-umbelliferone), trihydroxycinnamic acid derivatives (esculetin, methylesculetin, daphnetin), hydrocarbons (diphenylbutadiene, stilbene), dibenzalacetone, benzalacetophenone, naphthosulphonates (sodium salts of 2-naphthol-3,6-disulphonic acid and of 2-naphthol-6,8-disulphonic acid), organic benzophenone derivatives (2,4-dihydroxybenzophenone, 2,2′,4,4′-tetrahydroxybenzophenone, 2-hydroxy-4-methoxybenzophenone, 2-hydroxy-4-methoxybenzophenone-5-sulfonic acid, 2,2′-dihydroxy-4,4′-dimethoxybenzophenone, 2,2′-dihydroxy-4-methoxybenzophenone, disodium 2,2′-dihydroxy-4,4′-dimethoxy-5,5′-disulfobenzophenone), zinc oxide, titanium dioxide, and mixtures thereof.

Another preferred active material used in the composition of the present invention is a drug indicated for the treatment of fungal infections. Classes of drugs indicated for the treatment of fungal infections include synthetic triazole, ergosterol inhibitor, and polyene antifungal. Specific examples of drugs indicated for the treatment of fungal infections are itraconazole, ketoconazole, and amphotericin B.

Examples of vitamins that are available as active ingredients include, without limitation, vitamin A (retinol), vitamin D (cholecalciferol), vitamin E group (a-tocopherol and other tocopherols), vitamin K group ( phylloquinones and menaquinones), thiamine (vitamin B₁), riboflavin (vitamin B₂), niacin, vitamin B₆ group, folic acid, vitamin B₁₂ (cobalamins), biotin, vitamin C (ascorbic acid), and mixtures thereof. The amount of vitamin or vitamins present in the final encapsulated product of the present inventive subject matter is dependent on the particular vitamin and is generally the United States' Department of Agriculture Recommended Daily Allowances (USRDA) for that vitamin. For example, if vitamin C is the active ingredient and the encapsulated product is being used in a confectionery or chewing gum targeting adults, the amount of vitamin C in the encapsulated product would be 60 milligrams, which is the USRDA of vitamin C for adults.

Examples of minerals that are available as active ingredients include, without limitation, calcium, magnesium, phosphorus, iron, zinc, iodine, selenium, potassium, copper, manganese, molybdenum and mixtures thereof. As is the case with vitamins, the amount of mineral or minerals present in the final encapsulated product of the present inventive subject matter is dependent on the particular mineral and is generally the USRDA for that mineral. For example, if iodine is the active ingredient and the encapsulated product is being used in a confectionery or chewing gum targeting adults, the amount of iodine in the encapsulated product would be 150 micrograms, which is the USRDA of iodine for adults.

Examples of herbals that are available as active ingredients include, without limitation, echinacea, peppermint, licorice, goldenseal, panax pseudoginseng, grapeseed extract, bilberry, kava, ginko biloba, panax quinquefolium, Siberian ginseng, St. John's wort, bromelian, guglupids, hawthorn, garlic, ginger, angelica species, dandelion, goldenseal, and mixtures thereof. Further, examples of spices that are available as active ingredients include, without limitation, mustard, dillweed, cinnamon, garlic, black pepper, onion, sage, oregano, basil, cream of tartar, tarragon, cayenne pepper, red pepper, and mixtures thereof. This list of herbals and spices is for exemplary purposes and is not meant to be construed as limiting the inventive subject matter thereto.

Other active therapeutic agents commonly known as useful in the preparation of topical pharmaceutical compositions are further contemplated as within the scope of the present inventive subject matter.

The optimal pharmaceutical formulations will be determined by one skilled in the art depending upon considerations such as the route of administration and desired dosage. Such formulations may influence the physical state, stability, rate of in vivo release, and rate of in vivo clearance of the present therapeutic agents of the invention.

### Route(s) of Administration

The route(s) of administration of the compositions of the inventive subject matter are well known to those skilled in the art (see, for example, “Remington's Pharmaceutical Sciences”, 18th Edition, Chapter 86, pp. 1581-1592, Mack Publishing Company, 1990). The compositions may be administered orally, parenterally, by inhalation spray, topically, rectally, nasally, buccally, vaginally, or via an implanted reservoir in dosage formulations containing conventional non-toxic pharmaceutically-acceptable carriers, adjuvants, and vehicles. The term parenteral as used herein includes subcutaneous, intravenous, intramuscular, intraperitoneally, intrathecally, intraventricularly, intrasternal, and intracranial injection or infusion techniques.

The compositions may be administered in the form of sterile injectable preparations, for example, as sterile injectable aqueous or oleaginous suspensions. These suspensions, may be formulated according to techniques known in the art using suitable dispersing or wetting agents and suspending agents. The sterile injectable preparations may also be sterile injectable solutions or suspensions in non-toxic parenterally-acceptable diluents or solvents, for example, as solutions in 1,3-butanediol. Among the acceptable vehicles and solvents that may be employed are water, Ringer's solution and isotonic sodium chloride solution. In addition, sterile, fixed oils are conventionally employed as solvents or suspending mediums. For this purpose, any bland fixed oil such as a synthetic mono- or di-glyceride may be employed. Fatty acids such as oleic acid and its glyceride derivatives, including olive oil and castor oil, especially in their polyoxyethylated versions, are useful in the preparation of injectables. These oil solutions or suspensions may also contain long-chain alcohol diluents or dispersants.

Additionally, the compositions may be administered orally in the form of capsules, tablets, aqueous suspensions, or solutions. Tablets may contain carriers such as lactose and corn starch, and/or lubricating agents such as magnesium stearate. Capsules may contain diluents including lactose and dried corn starch. Aqueous suspensions may contain emulsifying and suspending agents combined with the active ingredient. The oral dosage forms may further contain sweetening, flavoring, coloring agents, or combinations thereof. Delivery in an enterically coated tablet, caplet, or capsule, to further enhance stability and provide release in the intestinal tract to improve absorption, is the best mode of administration currently contemplated.

The inventive compositions may also be administered rectally in the form of suppositories. These compositions can be prepared by mixing the drug with a suitable non-irritating excipient which is solid at room temperature, but liquid at rectal temperature and, therefore, will melt in the rectum to release the drug. Such materials include cocoa butter, beeswax, and polyethylene glycols.

Furthermore, the compounds may be administered topically, especially when the conditions addressed for treatment involve areas or organs readily accessible by topical application, including the lower intestinal tract. Suitable topical formulations can be readily prepared for such areas or organs. For example, topical application to the lower intestinal tract can be effected in a rectal suppository formulations (see above) or in suitable enema formulations.

It is envisioned that the continuous administration or sustained delivery of the compositions of the present invention may be advantageous for a given condition. While continuous administration may be accomplished via a mechanical means, such as with an infusion pump, it is contemplated that other modes of continuous or near continuous administration may be practiced. For example, such administration may be by subcutaneous or muscular injections as well as oral pills.

Techniques for formulating a variety of other sustained- or controlled-delivery means, such as liposome carriers, bio-erodible particles or beads and depot injections, are also known to those skilled in the art.

### Dosage

Appropriate dosage levels for the active therapeutic agents contemplated in the present inventive subject matter are well known to those of ordinary skill in the art. Dosage levels on the order of about 0.001 mg to about 5,000 mg per kilogram body weight of the active therapeutic compounds or compositions are known to be useful in the treatment of the diseases, disorders, and conditions contemplated in the present inventive subject matter. Typically, this effective amount of the active therapeutic agents will generally comprise from about 0.1 mg to about 100 mg per kilogram of patient body weight per day. Moreover, it will be understood that this dosage of active therapeutic agents can be administered in a single or multiple dosage units to provide the desired therapeutic effect. If desired, other therapeutic agents can be employed in conjunction with those provided by the present inventive subject matter.

The present inventive compositions may be given in a single or multiple doses daily. In a preferred embodiment, the present inventive compositions are given from one to three times daily. Starting with a low dose twice daily and slowly working up to higher doses if needed is a preferred strategy. The amount of active ingredients that may be combined with the carrier materials to produce a single dosage form will vary depending upon the host treated, the nature of the disease, disorder, or condition, and the nature of the active ingredients.

It is understood, however, that a specific dose level for any particular patient will depend upon a variety of factors well known in the art, including the activity of the specific compound employed; the age, body weight, general health, sex and diet of the patient; the time of administration; the rate of excretion; drug combination; the severity of the particular disorder being treated; and the form of administration. One of ordinary skill in the art would appreciate the variability of such factors and would be able to establish specific dose levels using no more than routine experimentation.

The optimal pharmaceutical formulations will be determined by one skilled in the art depending upon considerations such as the particular drug or drug combination and the desired dosage. See, for example, “Remington's Pharmaceutical Sciences”, 18th ed. (1990, Mack Publishing Co., Easton, Pa. 18042), pp. 1435-1712, the disclosure of which is hereby incorporated by reference. Such formulations may influence the physical state, stability, rate of in vivo release, and rate of in vivo clearance of the therapeutic agents.

### Drug Delivery Systems

In another embodiment, the inventive subject matter further relates to a drug delivery system comprising an LEA-1 protein and an active agent.

In an aspect of the inventive subject matter, said system is selected from the group consisting of a transdermal drug delivery system, an inhalation drug delivery system, an oral drug delivery system, and an implantable drug delivery system.

The inventive pharmaceutical compositions and delivery systems can be used to treat a wide variety of dermatological diseases, disorders, or conditions in a mammal, especially diseases affecting mammalian tissue. Examples of such diseases include, but are not limited to, eczema, infantile eczema, psoriasis, scalp psoriasis, atopic dermatitis, dermatitis herpetiformis, contact dermatitis, seborrheic dermatitis, neurodermatitis, pruritis, fungal diseases, and intertrigo.

### Methods for Improving the Quality and Storage Life of Frozen Biological Structures

The long-term storage of macromolecules such as proteins, nucleic acids, and polysaccharides, lipids, lipid micelles, monolayers, bilayers, artificial and biological membranes, live cells, and living organisms often relies upon sugars, for example sucrose, glycerol, and trehalose, to stabilize the structural integrity and thus improve viability upon freezing or lyophilized conditions. It is expected that LEA-1 protein will improve the effectiveness of cryoprotectant or lyophoprotectant formulations by reducing the formation of ice crystals, or reducing the rate of water removal during the transition to long-term dry storage conditions.

Thus, there is a need in the art to provide a method for the extended storage of biological material with no loss of the usability or quality of the material. Such a method would find broad applicability, for example, in the handling of foodstuffs, cut flowers, cells, tissues, gametes, organs, and whole organisms. The present inventive subject matter will be useful in the handling of all types of foodstuffs. The present inventive subject matter is particularly useful for the storage of fresh produce, fresh caught seafood, and meat, particularly prepared cuts of meat.

The high water affinity but semi-fluid structure of LEA-1 protein solutions, as well as their ability to help stabilize macromolecular structures, has many useful applications. For example, LEA-1 protein can be used in processes such as freezing, spray drying, freeze drying, and lyophilization where biological or pharmaceutical products require stabilization and retention of activity after desiccation or freezing. Additionally, LEA-1 protein can be incorporated into treatment of food products to reduce or prevent freezer burn during extended frozen storage conditions. This is expected to extend shelf life and improve the texture, taste, and appearance of all types of frozen prepared foods. LEA-1 protein also can stabilize treated products by inhibiting ice crystal formation. Food products could be sprayed with a solution of GmD-19, for example, as part of the freezing process.

The hydrophilic and high-degree of random coil structure of LEA-1 proteins also leads to their capacity to serve as water-binding proteins which can minimize water loss, act as hydration buffers to regulate water status, and interact with the surface of macromolecules as a water matrix, or as a water replacement, to resist protein denaturation or membrane phase changes in dehydrating tissues.

Thus, the present inventive subject matter relates to a method for improving the palatability of frozen food, comprising treating said food with an LEA-1 protein. Said treatment optionally occurs prior to, during, or after freezing.

Another method for improving the storage life of frozen vegetables and fruits also takes advantage of the properties of LEA-1 proteins. Plants may be genetically engineered to express an LEA-1 protein, then LEA-1 protein is expressed in plant tissues, providing endogenous protection of plant tissues which express the LEA-1 protein.

Thus, in another aspect of the inventive subject matter, said treatment is selected from the group consisting of genetically transforming said food to express an LEA-1 protein; and spraying, immersing, or injecting said food with an LEA-1 protein.

The present inventive subject matter further relates to a method for improving the storage life of frozen food, comprising treating said food with an LEA-1 protein. Said treatment optionally occurs prior to, during, or after freezing.

In an aspect of the inventive subject matter, said treatment is selected from the group consisting of genetically transforming said food to express an LEA-1 protein; and spraying, immersing, or injecting said food with an LEA-1 protein.

The present inventive subject matter further relates to a method for maintaining the integrity of a biological structure upon freezing, comprising treating said biological structure with an LEA-1 protein. Said treatment optionally occurs prior to, during, or after freezing.

In an aspect of the inventive subject matter, said biological structure is selected from the group consisting of a biological macromolecule, a lipid, a lipid micelle, a lipid monolayer, a lipid bilayer, an artificial membrane, a naturally-occurring biological membrane, a whole cell, a seed, an organ, and a whole organism.

In another aspect of the inventive subject matter, said biological macromolecule is selected from the group consisting of a polypeptide, a nucleic acid chain, and a polysaccharide.

In another aspect of the inventive subject matter, said treatment is selected from the group consisting of genetically transforming an organism to co-express an LEA-1 protein with said biological structure; and spraying, immersing, or injecting said biological structure.

In another aspect of the inventive subject matter, said method additionally comprises the step of adding one or more additional cryogenic compositions.

### Methods for Increasing Resistence to Environmental Stresses

In another embodiment, the inventive subject matter further relates to a method for increasing the resistence of an organism to drought stress, osmotic stress, heat stress, freezing stress, or a combination thereof, comprising transfecting said organism with an expression vector comprising an isolated GmD-19 DNA sequence operably linked to a promoter which is constitutively or inducibly expressed in said organism.

The supply of fresh water is decreasing in many farmed locations throughout the world, and there is a growing need to adapt cultivars for growth under dry, saline, or freezing conditions. A method for improving the drought, salinity, or freezing tolerance of plants has comprises genetically engineering plants for GmD-19 expression in vegetative tissues and reproductive tissues, and expressing GmD-19.

Thus, in an aspect of the inventive subject matter, said organism is a plant.

In a preferred embodiment, said plant is a cereal plant.

In a more preferred embodiment, said cereal plant is selected from the group consisting of wheat, corn, rice, barley, oat, rye, sorghum, and millet.

Preparation of LEA-1 Proteins The LEA-1 proteins of the present inventive subject matter may be readily prepared by standard techniques of molecular biology, utilizing techniques known to those of ordinary skill in the art, as described in greater detail herein. A representative LEA-1 protein of the present inventive subject matter may be readily prepared by standard biotechnological techniques, utilizing the following general synthetic pathway:

(i) isolating a cDNA molecule for an LEA-1 protein;

(ii) cloning said LEA-1 protein cDNA into an expression vector operatively linked to at least one control sequence compatible with a host cell or organism;

(iii) transforming said host cell or organism with said expression vector;

(iv) if said expression vector is not constitutively active, activating the expression of the LEA-1 protein; and

(v) purifying the LEA-1 protein from the host cell or organism.

The products and intermediates may be isolated or purified using one or more standard purification techniques known to one of ordinary skill in the art, including, for example, one or more of simple solvent evaporation, recrystallization, distillation, sublimation, filtration, polymerase chain reaction, Southern blotting, Northern blotting, Western blotting, chromatography, including thin-layer chromatography, affinity chromatography, gel filtration chromatography, ion exchange chromatography, FPLC, HPLC (e.g. reverse phase HPLC), column chromatography, flash chromatography, radial chromatography, trituration, salt precipitation, two-phase separation, polymer precipitation, heat denaturation, isoelectric separation, dialysis, and the like.

It should be recognized that one skilled in the art may vary the exemplary methods as set forth herein. Such variations are expected to be within the scope of the inventive subject matter.

### EXAMPLES

The following examples are illustrative of the present inventive subject matter and are not intended to be limitations thereon. Unless otherwise indicated, all percentages are based upon 100% by weight of the final composition. All starting materials, reagents, and solvents were commercially available and were used either as obtained from chemical suppliers, synthesized according to known literature procedures, and/or washed, dried, distilled, recrystallized, and/or purified before use.

### SUMMARY OF EXAMPLES

In order to confirm the structural and physicochemical characteristics of group 1 LEA from soybean in detail, high milligram quantities of the recombinant soybean GmD-19 protein were produced and purified from E. coli. Affinity tag sequences were removed from the pET30a expression vector in order to express the protein.

Initial purification took advantage of the unique property of GmD-19 solubility following exposure to boiling conditions. Subsequent purification steps included preparative isoelectric focusing (IEF) and anion exchange chromatography, which were needed if one desired a high degree of purification. To determine whether the GmD-19 structure was altered by heat denaturation, the GmD-19 protein was also purified without boiling. Precipitation at 60% (NH₄)₂SO₄ was substituted for heat and used as the initial purification step. Preparative IEF and anion exchange column chromatography steps followed. However, to attain homogeneity, it was necessary to include an additional cation exchange column chromatography step.

Both rGmD-19s purified using heat and (NH₄)₂SO₄ showed identical electrophoretic mobility following analysis by native or denaturing SDS-polyacrylamide gel electrophoresis (SDS-PAGE). Also, DSC scans of proteins at high temperatures showed no denaturing events at the extraction temperatures, unlike control proteins such as BSA. The identity of the proteins purified by either purification strategy was confirmed by matrix-assisted laser desorption/ionization-time of flight (MALDI-TOF) MS. The molecular mass of GmD-19 was 11.359 kDa, which is identical to the predicted molecular weight, without -terminal fMet, 11.359 kDa. The purified proteins tested negative for sugars and sugar alcohols. Both non-induced E. coli cells and cells induced for GmD-19 overexpression showed similar growth rates, suggesting that GmD-19 protein accumulation was not harmful to cell viability.

### Example 1

### Cloning and Expression of Soybean Group 1 LEA

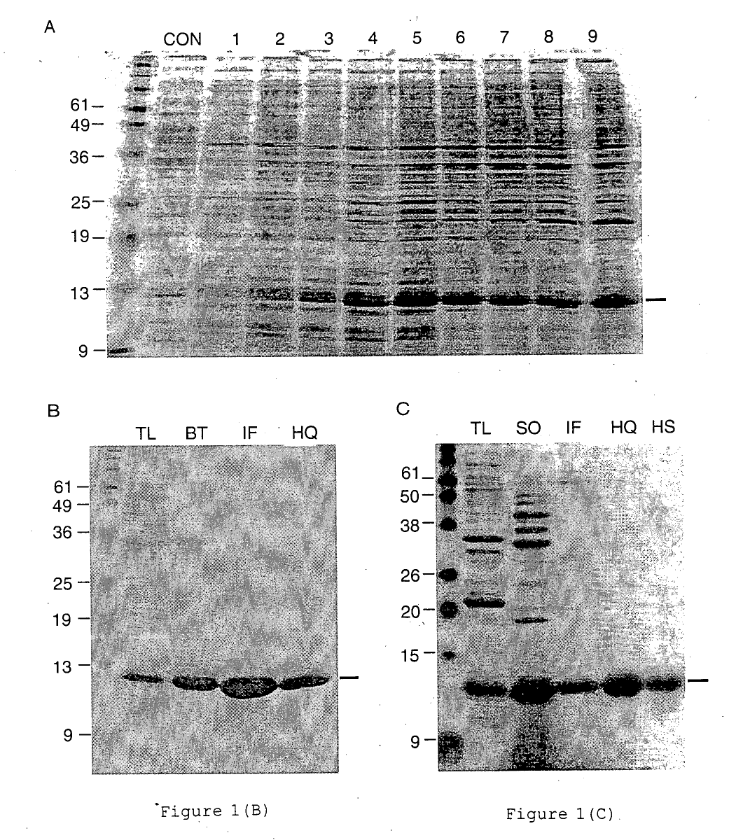

The soybean group 1 LEA cDNA, GmD-19, coding region of 321 bp, Accession Nos. U66317 and AAB68027, was amplified using DNA polymerase and gene-specific primers containing NcoI or EcoRI restriction sites. The amplified fragment was digested with NcoI/EcoRI and ligated into the NcoI/EcoRI sites of the pET30a E. coli expression vector. His- and S-tag sequences, 157 bp, were then removed from the cloning vector by inverse PCR using Pfu polymerase and outward facing primers containing AscII restriction sites. The amplification product was digested with AscII and religated with T4 ligase. The integrity of the cloned insert was confirmed by automated DNA sequencing. The resulting pET30a::GmD-19 was introduced into E. coli BL21 plysS cells and grown in 2× YT medium under kanamycin selection at 50 μg/ml, at 37° C. with vigorous agitation at 300 rpm. Recombinant GmD-19 protein expression was induced by adding IPTG to a final concentration of 0.1 mM when cells reached an OD600=0.8. Cells were harvested when the culture reached an OD600=1.5. Expression of the recombinant protein GmD-19 was confirmed by 15% SDS-PAGE. In FIG. 1(A), rGmD-19 accumulation is shown for different times following IPTG induction. Cells were collected at various time points and soluble proteins were extracted. Con=control, no IPTG; lane 1, induction point 120 min; lane 2, 135 min; lane 3, 150 min; lane 4, 180 min; lane 5, 210 min; lane 6, 240 min; lane 7, 270 min; lane 8, 300 min; lane 9, 360 min. Approximately 10 μg of protein was loaded in each lane.

### Example 2

### Purification of GmD-19 from E. coli

Bacterial cells were harvested by centrifugation at 5000 g and resuspended in B-PER Bacterial Extraction Buffer in the presence of a protease inhibitor cocktail. The soluble cell lysate extract was sonicated to reduce viscosity, denatured in boiling water for 10 min., and clarified by centrifugation at 26,500×g for 20 min. The clarified supernatant was concentrated using a polycarbonate centrifugal filter and dialyzed overnight against 10 mM Tris-HCl, pH=7.5, using dialysis membrane tubing. The dialyzed extracts were subjected to preparative IEF in the presence of ampholytes at 2% v/v, pH range=5-7, at 15W constant power for 4 hours. Fractions were surveyed by 12% SDS-PAGE and fractions containing rGmD-19 protein were pooled and stored at −20° C.

Further purification of the pooled IEF fractions was conducted using anionic exchange column chromatography on a High-Q column in 10 mM Tris-HCl, pH 7.5 eluted by a 0-250 mM NaCl gradient at 1 ml/min flow rate. Collected fractions, 2 ml, were analyzed by SDS-PAGE, pooled, and stored as the final purified proteins.

Alternatively, rGmD-19 was purified without heat denaturation by 60% ammonium sulfate precipitation. Clarified supernatant was recovered and desalted using a polycarbonate centrifugal filter and dialyzed prior to preparative isoelectric focusing (IEF) and anion exchange column chromatography. Cation exchange column chromatography using a High-S column equilibrated with 10 mM Na-acetate, pH 4.8, was used as a final purification step. rGmD-19 was eluted using a 0-500 mM NaCl gradient at 1 ml/min flow rate and fractions containing the purified protein were pooled and desalted by dialysis as described above.

In total cell lysates of E. coli BL21 plysS cells, the putative rGmD-19 protein with an apparent molecular weight of 11.4-kDa was induced. FIG. 1(B) shows the results after subsequent purification steps including preparative IEF and anion exchange chromatography. A high degree of purification was obtained. In FIG. 1(B), composite purification steps of rGmD-19 treated with boiling are shown. Approximate amount of protein loaded in each lane is indicated in parentheses. TL, Total lysate (10 μg); BT, Boiling treated (5 μg); IF, Isoelectric focusing (2 μg); HQ, High Q (1.5 μg).

FIG. 1(C) shows the results composite purification steps of rGmD-19 without boiling. TL, Total lysate (10 μg); SO, 60% (NH₄)₂SO₄ salting-out (10 μg); IF, Isoelectric focusing (2 μg); HQ, High Q (2 μg); HS, High S (1.5 μg). Line designates rGmD-19. The relative masses of prestained or stainable molecular weight standards are designated in kilodaltons.

### Example 3

### Thermal Stability of rGmD-19 by Differential Scanning Calorimetry (DSC) and Thermal Analysis

Three mg of lyophilized protein powder were weighed on a DSC volatile sample pan and hydrated at 80% relative humidity (RH) controlled by saturated KCl at room temperature. Thermal events were measured from 0° C. to 100° C. at a rate of 10° C./min using a differential scanning calorimeter calibrated for temperature using methylene chloride at −95° C. and indium at 156° C. as standards and for energy with indium at 28.54 Jg⁻¹. Helium gas was used for purging at a rate of 20 ml/min. To standardize the dry mass of each sample, heat flow in every DSC scan was divided by the sample dry weight.

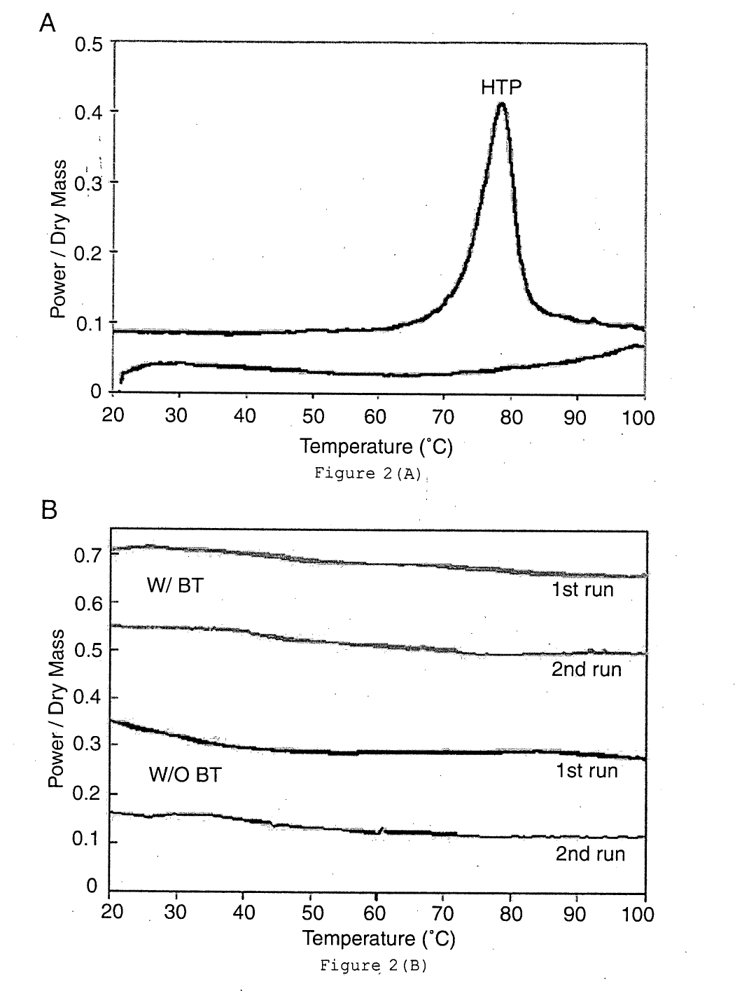

Differential scanning calorimetry (DSC) was used to investigate a potential cooperative structural transition of GmD-19. FIGS. 2(A) and 2(B) display heating thermograms of BSA and rGmD-19 using differential scanning colorimetry (DSC). DSC heat scans with and without boiling treatment (BT) were performed at 10° C./min from 0° C. to 100° C. Plots of each scan are offset slightly for clarity. “HTP” designates the high temperature peak. The DSC scans in FIG. 2(A) showed a typical denaturation pattern of a heat-labile protein, e.g., BSA undergoes thermal denaturation around 80° C. In contrast, as shown in FIG. 2(B), DSC scans of GmD-19, purified with or without boiling, showed no detectable HTP. This result showed no evidence of structural rearrangements when the protein was heated to high temperatures. Similar results have been reported for a group 1 LEA protein isolated from pea embryonic axes. These DSC results are also consistent with previous studies indicating a low proportion or a lack of distinct tertiary structure in group 1 LEA proteins. Even though heating may alter the structure of GmD-19 on a molecular scale, such changes may be of low cooperativity and thus not detectable by DSC. Therefore, spectroscopic methods, more sensitive at detecting molecular scale changes in structure, were employed.

### Example 4

### UV-absorption Spectroscopy

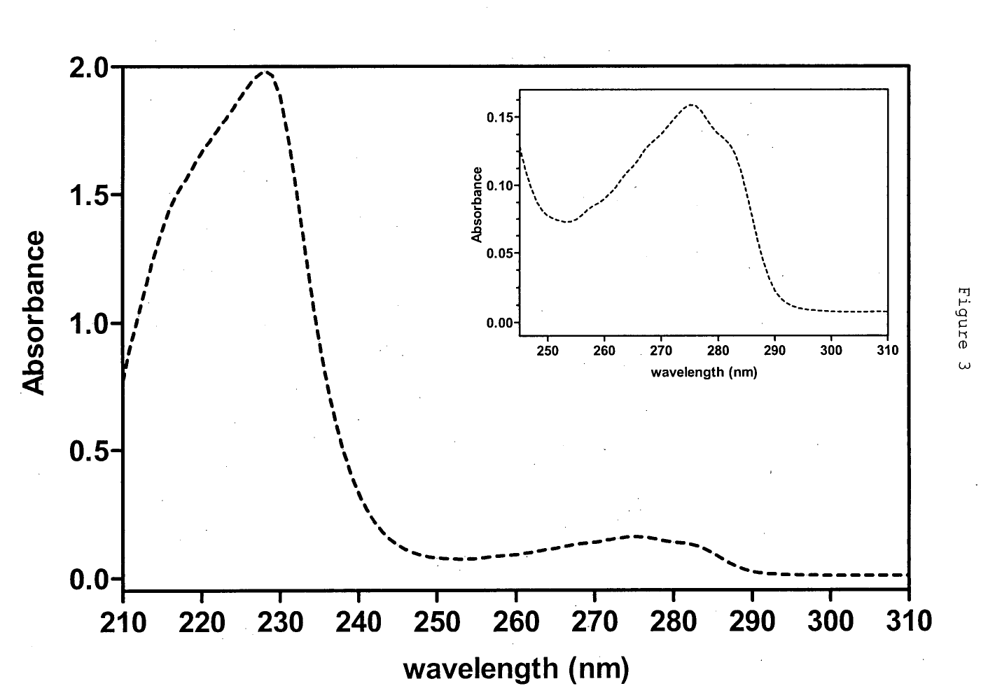

UV absorption spectra were recorded with a diode array spectrophotometer. The concentration of GmD-19 was calculated from the absorbance of the samples at 280 nm in the presence of 6M guanidinium-HCl (eTyr=1,285 cm⁻¹M⁻¹). FIG. 3 shows UV-absorption spectrum of rGmD-19, 77 μM, in 50 mM phosphate buffer, pH=7, 24° C. The inset shows the expanded near-UV region of the spectrum. Second derivatives were calculated by the Savitzky-Golay differentiation technique using a filtering length of 9. The temperature dependence of the difference between the second derivatives at 283 nm and 279 nm was determined in 50 mM phosphate buffer solutions. The sample temperature was modified and controlled by a temperature-controlled cell holder.

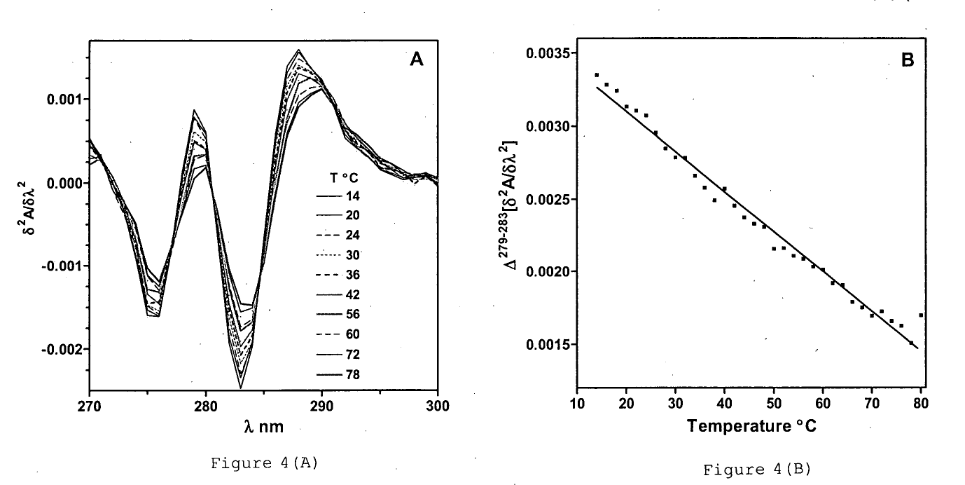

Second derivative UV-absorption spectroscopy is a reliable, quantitative and sensitive tool to study the hydration of aromatic residues. Using tyrosine fluorescence spectroscopy, it has been reported that there is no difference in the tyrosine environment of a group 1 LEA protein from pea before and after heat treatment at 80° C. In contrast to this earlier work, FIG. 4(A) shows the spectral changes that occur when the temperature was increased from 10° C. to 80° C. In FIG. 4(A), the spectra shown, reading downward at 283 nm, represent increasing temperatures from 14-78° C. The temperatures are indicated in the figure. In the spectral region displayed, the temperature-induced changes observed in the second derivative spectrum of GmD-19 reflect the changes in the hydration of the Tyr residue.

Even though the Tyr residue was highly hydrated at 10° C., these spectral changes indicate that the protein becomes more hydrated as the temperature increased. These changes suggest that heating promotes a structural transition involving an unfolding process. In addition to the changes in the intensity of the second derivative spectra, another important feature observed in this study is the presence of several isobestic points, as shown in FIG. 4(A) . The presence of an isobestic point indicates a transformation involving two components. The relative proportion of the two conformational states is fairly constant with temperature in the 12-80° C. range, as evidenced by unchanging isobestic points. Because second derivative spectra obey Beer's law, as well as zero order spectra, the difference between the second derivative values at any two given wavelengths is a measure of the relative change in the population of the conformational states. We have done this using the values of the derivative at 279 nm and 283 nm. FIG. 4(B) shows that between 12° C. and 80° C., where the temperature-induced changes are represented using the difference between the derivative values at 279 nm and 283 nm there is a continuous change in Δ(279-282), δ²ε/δλ². Consistent with the DSC results, the slope of the plot suggests that the cooperativity of this conformational transition is quite low.

### Example 5

### Water Sorption Isotherms

The hydration properties of the recombinant LEA proteins were evaluated using water sorption isotherms. In a parallel fashion, we compared the sorption profile of LEA proteins with a water-soluble protein, BSA, and a seed storage protein, gluten. Three to five mg of each protein were weighed using a microbalance and loaded onto a Differential Scanning Calorimeter (DSC) volatile aluminum sample pan or resealable pan. To control the humidity from 0 to 100% RH, we placed samples in airtight jars containing different saturated salt solutions. At high RH's, constant weight, assumed to be equilibrium, was achieved within 1-2 hours. At low RH's, there were no measurable changes in weight after 1-2 days, though changes may have been sufficiently slowed to limit detection. “Equilibrated” samples were hermetically sealed and fresh weights were obtained. To obtain dry weights, pans were punctured and placed at 95° C. for 24 hours before weighing.

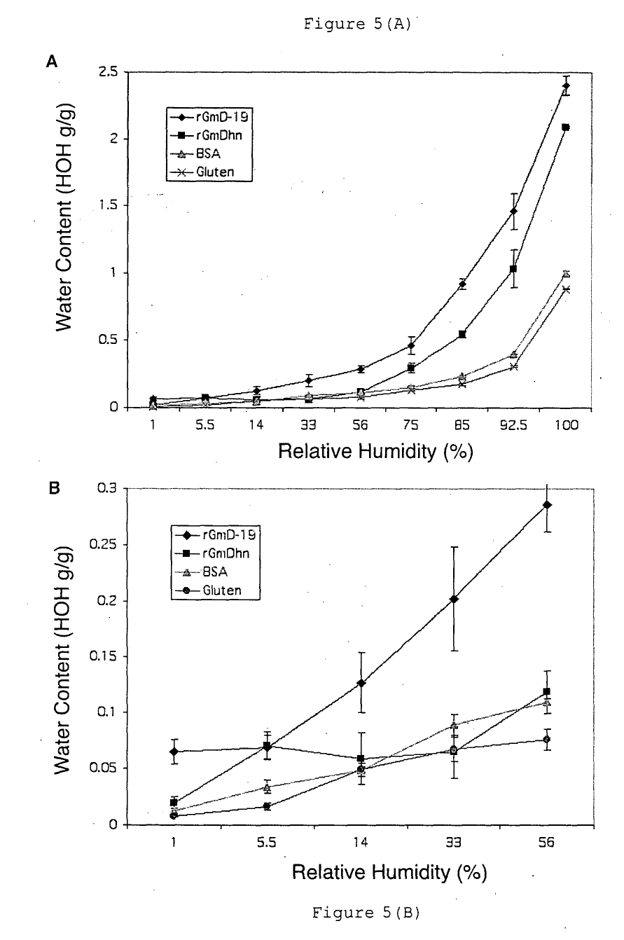

Due to their highly hydrophilic amino acid sequences, group 1 and 2 LEA proteins were predicted to have strong or many interactions with water. As shown in FIG. 5, the relationship between RH and water content describes some of these interactions in LEA proteins and was compared with that of other proteins under constant temperature. FIG. 5(A) shows water absorption from the entire RH range tested. FIG. 5(B) shows detail of water absorption at low moisture levels.

We also compared the water sorption of rGmD-19 and rGmDhn with BSA and gluten. Purified recombinant rGmD-19 and rGmDhn absorb water very rapidly. Both LEA proteins were solubilized within a few minutes in >91% RH chamber, compared to BSA and gluten that did not dissolve at this RH. Overall, as shown in FIG. 5(A), the water content of all four proteins increased as relative humidity increased. However, each protein showed highly diverse patterns of hydration rate and capacity. BSA and gluten showed similar hydration patterns through the entire relative humidity range. These proteins adsorbed water slowly, lost it quickly, and had water content-RH relationships typical of water-soluble proteins.

In contrast, LEA proteins showed much steeper hydration rates, dried relatively slowly, and achieved very high water contents at high RH. As shown in FIG. 5(B), recombinant GmD-19 retained water at low RH, giving it unusually high water contents under extremely dry conditions and demonstrating its higher water-holding capacity compared to other proteins tested. At high RH, hydration patterns of LEA proteins were similar to that of sucrose glasses in that the proteins dissolve quickly and continue to absorb large amounts of water. In particular, group 1 LEA rGmD-19 showed the highest hydration level throughout the entire RH range. Under 100% RH, water bound to rGmD-19 is approximately 2.5 times that bound to BSA and gluten. At low RH, hydration patterns of LEA proteins are dissimilar to that of sucrose in that they reach an equilibrium water content faster and this water content remains high. In particular, as shown in FIG. 5(B), the water content of rGmD-19 is greater than other molecules and mixtures studied.

### Example 6

### DSC and Thermal Analysis

The thermal behavior of LEA, BSA, and gluten mixed with different amounts of water were analyzed using a Differential Scanning Calorimeter according to methods previously reported for seeds or pollen. Temperature and enthalpy or heat capacity changes in first or second order transitions, respectively, were recorded between −150° C. and 50° C., with samples cooled and heated at a rate of 10° C./min and instrument settings as described in Example 3. Each scan has been standardized against dry mass. Absolute water content of the protein in each scan was designated on scans. Melting transitions were identified by the existence of endothermic peaks. Melting transitions, first order transitions, were detected as peaks in the scans, and glass transitions, second order transitions, were detected as baseline shifts.

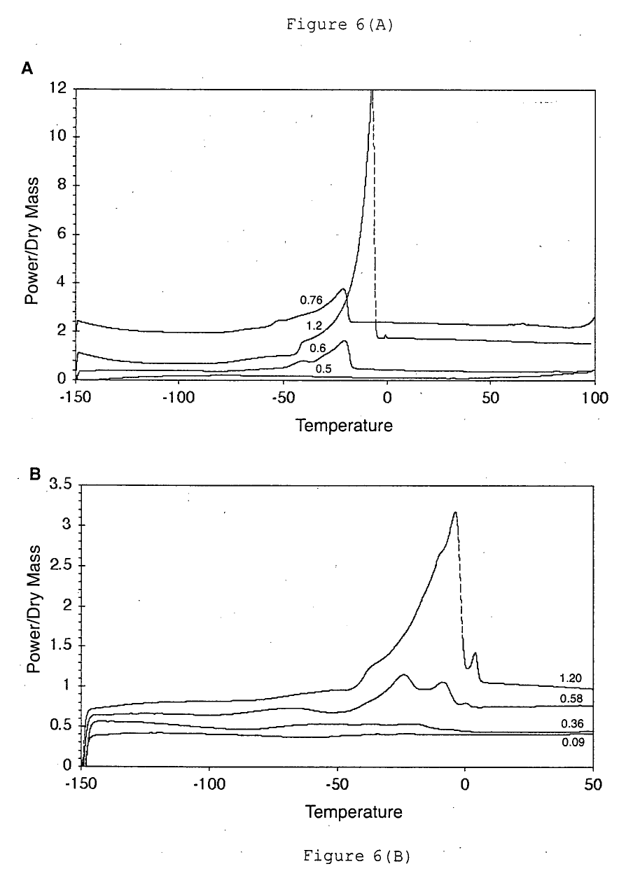

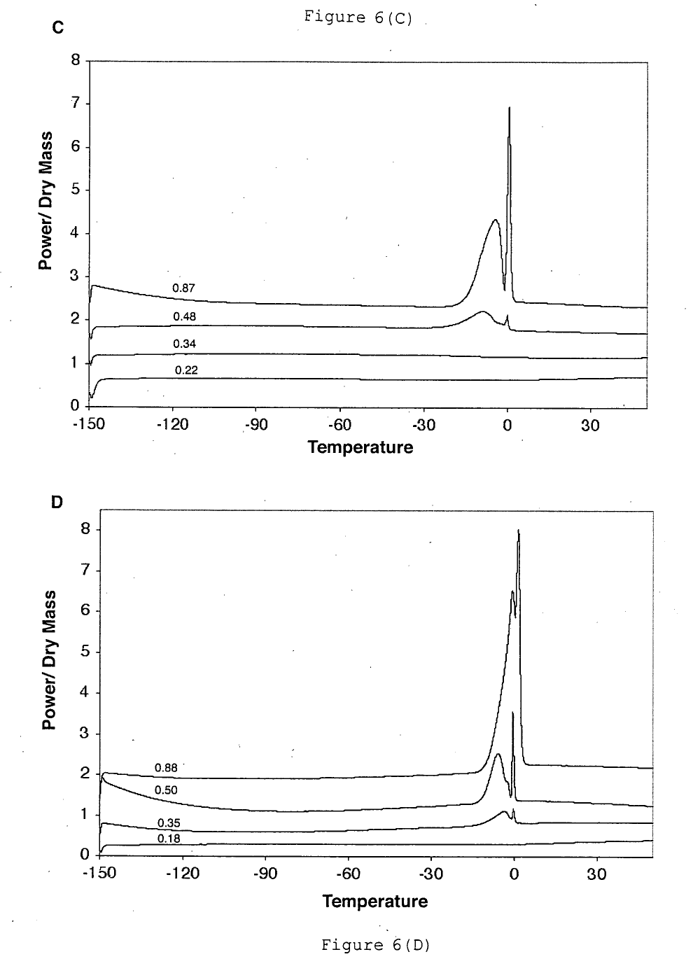

Calculations of changes in enthalpy (ΔH) and heat capacity (ΔCp) and onset temperatures Tₘₑₗₜ, T_{glass}, or T_{g} were made using the installed software. Changes in transition characteristics were measured as a function of the water content of the protein, which was manipulated using RH chambers as described in Example 5. As shown in FIGS. 6(A) and 6(B), the water content at which freezing transitions were limited, i.e. unfrozen water content, was determined from the x-intercept of the linear regression between enthalpy of melting transition (ΔH/g dw) and water content of the sample (g H₂O/g dw) according to published procedures. Enthalpy of each melting transition was linear-regressed against water content. The water contents at which freezing and melting transitions were not observable under the conditions of the experiment were calculated by x-intercept.

LEA proteins had thermal behaviors that were different from other water-soluble proteins. Melting transitions were complex showing several low temperature peaks, and the size of transitions increased with increasing water content. Unlike thermograms from BSA or gluten, thermograms from LEA proteins lacked a sharp melting peak at 0° C., indicating that phase separations leading to the formation of pure water crystals did not occur. The temperatures of the melting transitions in LEA proteins were much lower than observed in BSA or gluten at all water contents, demonstrating that LEA proteins exhibited non-colligative behavior and strong water-solute interactions typical of cryoprotected cells.

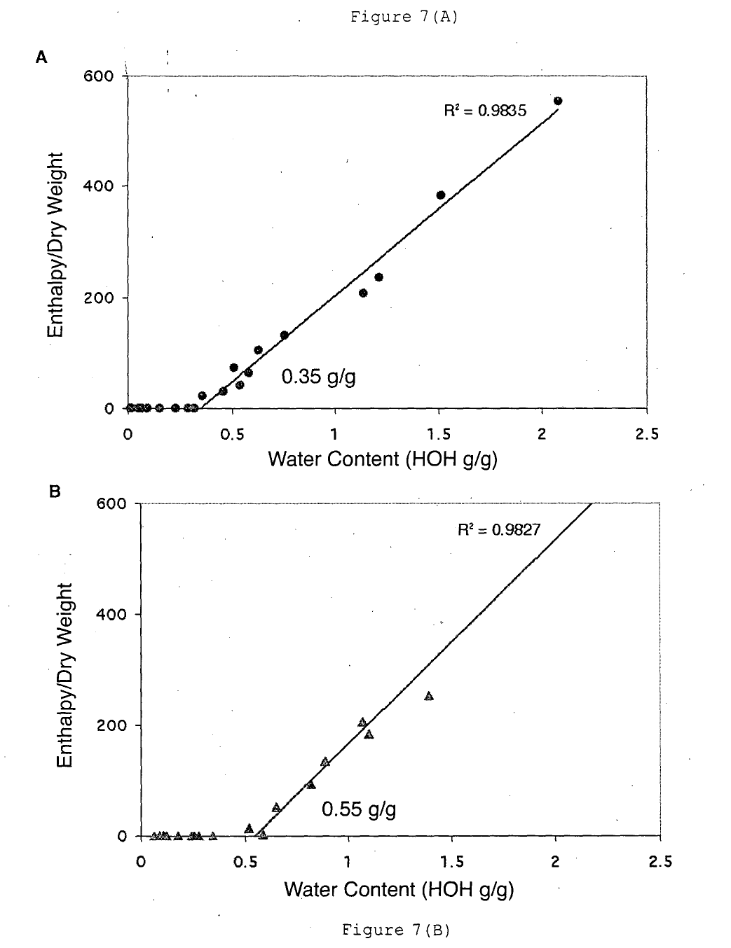

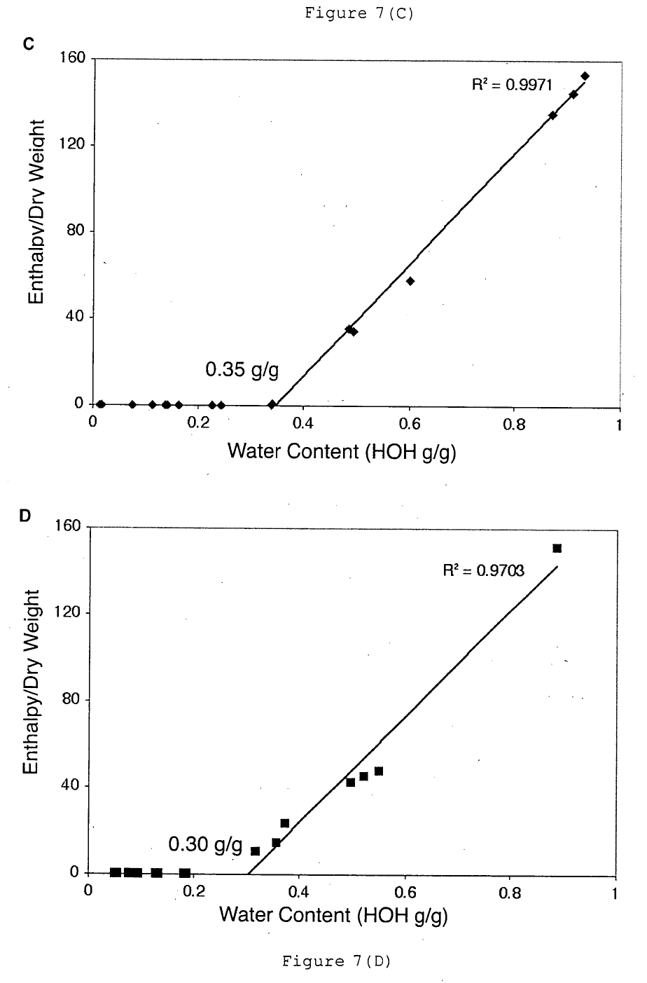

FIGS. 7(A)-7(D) show the unfrozen water contents of A) rGmD-19, B) rGmDhn, C) BSA, D) Gluten. As shown in FIG. 7, the water content below which ice formation was restricted, i.e. unfrozen water content, was 0.35 g/g for BSA, gluten, and GmDhn, which is typical for seeds and pollen. The unfrozen water content for GmD-19 was 0.55 g/g, and the higher value compared to other systems indicates that this protein had greater capacity to alter molecular motions of the aqueous environment sufficiently to prevent ice formation.

The LEA proteins also have unusual glass-forming properties compared to other systems studied. Typically glass transition temperatures increase as molecular weight of the solute increases; and accordingly, one would expect LEA proteins to have high-glass transition temperatures, as is observed for BSA and gluten, as well as seeds, which contain high quantities of LEA proteins. In contrast, both GmDhn and GmD-19 have low T_{g} at low water contents, even when compared to glucose. At higher water contents, glass-to-liquid transitions are easily detectable, unlike BSA, suggesting the formation of relatively stable glassy states; and T_{g} values are relatively high, intermediate between sugar solutions and PVP. Though T_{g}-water content relationships are similar for rGmDhn and rGmD-19, the size of the baseline shift during glass transitions is largest for rGmD-19, intermediate for rGmDhn and gluten, and small for BSA. This suggests that more water molecules participate in the aqueous rGmD-19 glass, furthering the idea that rGmD-19 has long-range forces that restrict the molecular mobility of water in solution. These observations are in contrast to those recently made for a group 3 LEA protein from Typha latifolia pollen D-7. D-7 was shown to increase T_{g} in protein-sucrose mixtures, an observation that is the opposite of our observation that T_{g} occurs at a relatively low temperature, which is why rGmD-19 is fluid even when dry.

Recombinant GmD-19s high affinity for water was demonstrated by relatively high water contents in sorption isotherms, high unfrozen water content in melting transitions and large changes in heat capacity during glass-to-liquid transitions. The greater water affinity for group 1 LEA rGmD-19 compared to group 2 LEA rGMDhn may result from greater random coil formation and the presence of extended substructure. In spite of the higher affinity for water, aqueous solutions of LEA proteins remain relatively fluid, as evidenced by the relatively low T_{g} at low water contents. This unexpected behavior suggests that LEA proteins facilitate the formation of amorphous states, but do not readily form amorphous solid states. The thermal and sorption behaviors of recombinant soybean group 1 and 2 proteins suggest that they affect the physical state of water in different ways. Previous studies suggested that mixtures of LEA-like proteins could control the rate and amounts of water sorption and drying, but only in the presence of high sugar concentrations. Here, we have shown that LEA proteins have properties that are distinct from both sugars and other water-soluble proteins, that these properties are independent of the presence of sugars, and that they are more strongly expressed in group 1-type LEA proteins.

In conclusion, our results demonstrate that GmD-19 has a high affinity for water, but only partially restricts the molecular mobility of water molecules.

### Example 7

### Production of GmD-19 in Plants

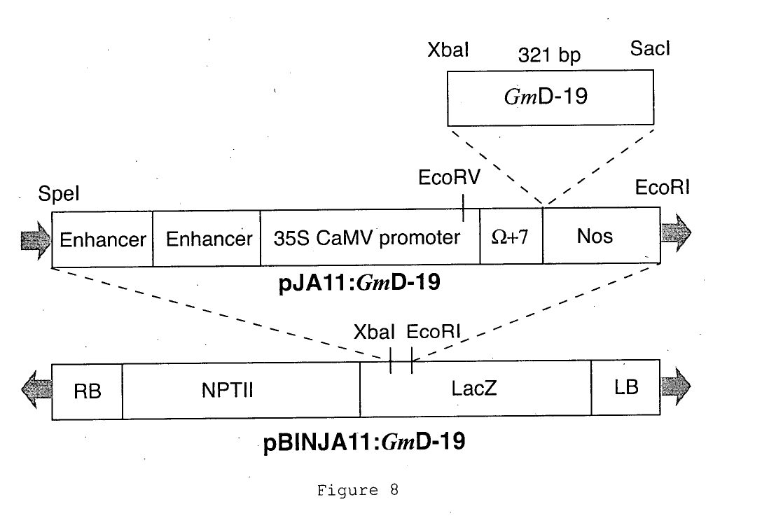

Arabidopsis plants were transformed with a T-DNA vector, pBIN19, constitutively expressing the GmD-19 cDNA under the control of an enhanced CaMV 35S expression cassette to boost transgene expression using an Agrobacterium-mediated in planta vacuum infiltration procedure. FIG. 8 is a schematic of the pBIN19 T-DNA vector for constitutively expressing the GmD-19 cDNA under the control of the enhanced CaMV 35S expression cassette.

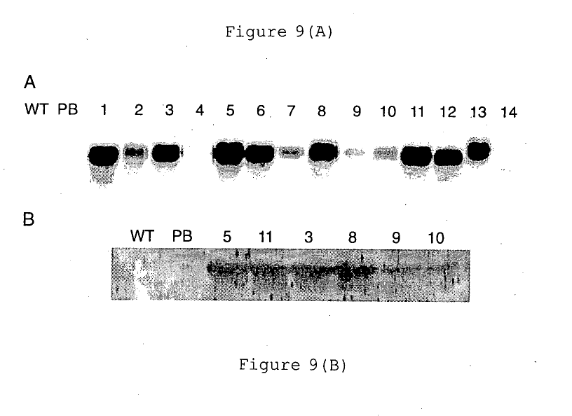

Following screening of approximately 2500 T1 progeny, 14 independent, kanamycin transformants were found to carry the GmD-19 transgene. Transgene integration was confirmed by PCR and genomic Southern blotting. In addition to detection of two bands representing the two Arabidopsis group 1 LEA genes AtEm1 and AtEm6 in wild-type and pBIN19 control lines, 1-5 copies of the soybean transgene insert were observed depending on the Arabidopsis line tested. FIG. 9(A) shows steady state accumulation of the 0.75 kb transcript in wild-type (WT), pBIN19 (PB) and independent transgenic Arabidopsis lines (T3) (numbered 1-14) on 1.2% agarose electrophoresis, electroblotted onto a nylon membrane and hybridized with full-length GmD-19 cDNA insert. No transgene was detected in wild-type or pBIN19 control lines lacking the cDNA insert. There was no correlation between gene copy number and transcript abundance. However, transgenic lines of generation T2 displayed varying degrees of accumulation of the 0.75 kb GmD-19 transcript from no detectable, shown in lines 4 and 14, to highly abundant transcript accumulation, shown in lines 5 and 11 of FIG. 9(A).

FIG. 9(B) shows protein accumulation in WT, PB and T3 lines as resolved by 15% SDS-PAGE, electroblotted to a PDVF membrane and immunologically detected using anti-Em (wheat) antibody.

Six transgenic lines that displayed 3:1 segregation ratios for kanamycin resistance were selected for further analysis based on their GmD-19 mRNA accumulation patterns. Selected lines were categorized as high, shown in lines 5 and 11, medium, shown in lines 3 and 8, or low expressers, shown in lines 9 and 10 of FIG. 9(A). Western blot analysis verified protein accumulation in these selected lines in the T3 generation, which correlated well with transcript accumulation shown in FIG. 9(B). A single 11.5 kDa protein accumulated in transgenic lines consistent with the predicted molecular weight of 11.49 kDa for GmD-19. Compared to known quantities of purified rGmD-19 protein, plant accumulation of GmD-19 was relatively low, <0.1% of total leaf protein, even in lines that accumulated the highest protein amounts, shown in lines 5 and 11. However, all transgenic plants, even the high-expressing lines, displayed normal phenotypes.

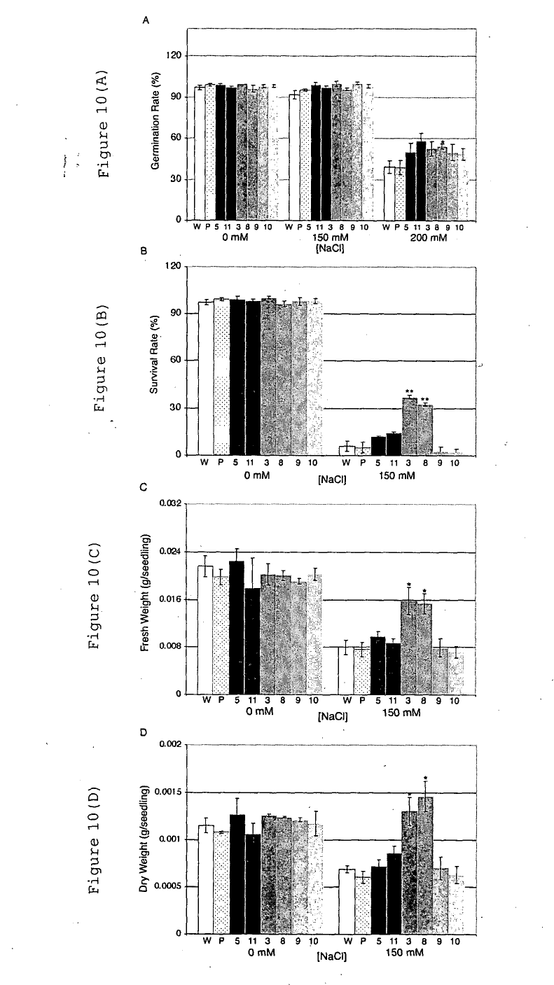

FIGS. 10(A)-10(D) summarize germination, survival, fresh weight, and dry weight of GmD-19 transgenic lines following chronic exposure at 0, 150, and 200 mM NaCl in agar plates. FIG. 10(A) shows the percentage of seed germination scored after 21 days. As shown in FIG. 10(A), plants with a medium number of GmD-19 copies, for example in line 8, showed improved germination rates compared to controls. FIG. 10(B) shows the percentage of survival scored after 31 days. Non-bleached, actively growing plants were scored as viable. FIG. 10(C) shows the mean fresh weight of individual seedlings, and FIG. 10(D) shows the mean dry weight of individual seedlings. Statistical significance compared with the values of wild-type, was determined by the Student T-test (two-tailed test) at p<0.05 and p<0.01 as indicated on individual bars by (*) and (**), respectively. Also under chronic exposure to 150 mM NaCl, the two medium-expressing lines showed statistically improved survival rates in FIG. 10(B), and higher fresh and dry weights in FIGS. 10(C) and 10(D).

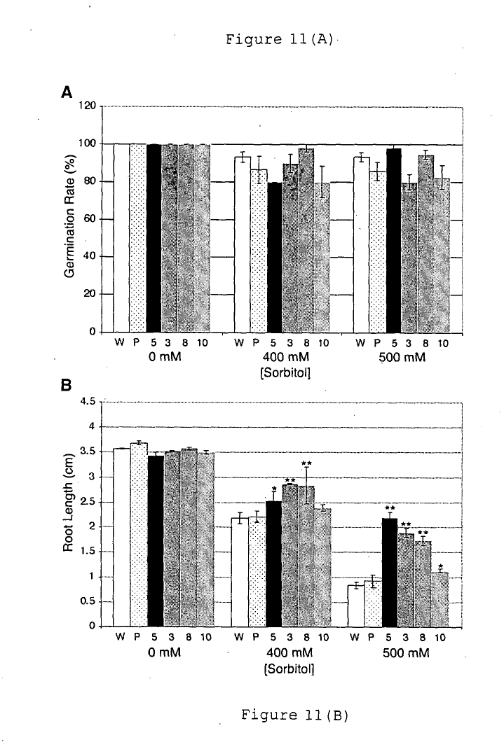

FIGS. 11(A) and 11(B) summarize germination and root length of GmD-19 transgenic lines under osmotic stress in wild-type, pBIN19, and selected transgenic lines expressing the transgene at high (line 5), medium (lines 3 and 8), and low (line 10) amounts following chronic exposure to 0, 400 and 500 mM sorbitol, respectively, in agar plates. As shown in FIG. 11(A), which shows the percentage of seed germination after 21 days, scored as positive if both radical emergence and cotyledon expansion occurred, at high molar stress using 500 mM sorbitol, the enhancement of root growth showed a good correlation with GmD-19 protein accumulation. FIG. 11(B) shows mean root length of individual seedlings. Means of 3 replicate trials containing 10 seeds for each line + standard error were plotted with darker shaded bars correlated with greater transgene expression. Medium-expressing line 8 was less sensitive to freezing stress than were wild-type and pBIN19 control lines. These data indicate that plants producing GmD-19 can be grown under long-term salinity or drought exposure that are likely to occur under field conditions.

Applicants note that additional experimental details, such as identification of the source/manufacturer of materials and reagents, and primer and probe sequences, are found in U.S. Patent Application No. 60/403,329 and Applicants' publication, Temperature-Induced Extended Helix/Random Coil Transitions in a Group 1 Late Embryogenesis-Abundant Protein from Soybean, Jose L. Soulages, Kangmin Kim, Christina Walters, and John C. Cushman, Plant Physiology, 128:822-832 (March 2002), the contents of which are incorporated by reference herein in full.

The inventive subject matter being thus described, it will be obvious that the same may be modified or varied in many ways. Such modifications and variations are not to be regarded as a departure from the spirit and scope of the inventive subject matter and all such modifications and variations are intended to be included within the scope of the following claims.

We claim:

1. A method for increasing the water absorbency or water retention capacity of a substrate material, said method comprising the steps of:
(i) combining an LEA-1 protein with said substrate material to form a mixture; and
(ii) forming said mixture into a configuration in which said LEA-1 protein contacts water molecules upon exposure to an environment comprising said water molecules.

2. The method of claim 1, wherein said water molecules are selected from the group consisting of water vapor, liquid water, or ice.

3. The method of claim 1, wherein said substrate material is selected from the group consisting of paper products, cloth or fabric, hydrogel, foam, resin, and polymer matrix.

4. The method of claim 1, comprising the additional step of binding said mixture together to form substantially water-insoluble bonds between said LEA-1 protein and said substrate material.

5. An absorbent product produced by the process of:
(i) combining a first component comprising an LEA-1 protein with a composition to form a mixture; and
(ii) forming said mixture into a configuration in which said LEA-1 protein contacts water molecules upon exposure to an environment comprising said water molecules.

6. The product method of claim 5, wherein said water molecules are selected from the group consisting of water vapor, liquid water, or ice.

7. The product of claim 5, wherein said substrate material is selected from the group consisting of paper products, cloth or fabric, hydrogel, foam, resin, and polymer matrix.

8. The product of claim 7, selected from the group consisting of wound dressings, disposable diapers, adult incontinence pads, adult incontinence briefs, menstrual pads, sanitary napkins, and tampons.

9. A pharmaceutical composition comprising:
(i) an active therapeutic agent;
(ii) an LEA-1 protein; and
(iii) a pharmaceutically acceptable carrier.

10. A drug delivery system comprising an LEA-1 protein and an active agent.

11. The drug delivery system of claim 10, wherein said system is selected from the group consisting of a transdermal drug delivery system, an inhalation drug delivery system, an oral drug delivery system, and an implantable drug delivery system.

12. A method for maintaining or increasing the hydration state of a pharmaceutical or cosmetic composition, said method comprising combining an LEA-1 protein with said pharmaceutical or cosmetic composition.

13. The method of claim 12, wherein said composition is a topical formulation.

14. The method of claim 13, wherein said topical formulation is a transdermal drug delivery formulation.

15. The method of claim 12, wherein said composition is a sustained or controlled drug release system.

16. The method of claim 12, wherein said composition is a pharmaceutical or cosmetic gel, cream, lotion, ointment, soap, shampoo, solution, spray, jelly, gel, emulsion, wound dressing, suppository, or artificial skin membrane.

17. A method for improving the palatability of frozen food, comprising treating said food with an LEA-1 protein.

18. The method of claim 17, wherein said treatment is selected from the group consisting of genetically transforming said food to express an LEA-1 protein; and spraying, immersing, or injecting said food with an LEA-1 protein.

19. A method for improving the storage life of frozen food, comprising treating said food with an LEA-1 protein.

20. The method of claim 18, wherein said treatment is selected from the group consisting of genetically transforming said food to express an LEA-1 protein; and spraying, immersing, or injecting said food with an LEA-1 protein.

21. A method for maintaining the integrity of a biological structure upon freezing, comprising treating said biological structure with an LEA-1 protein.

22. The method of claim 21, wherein said biological structure is selected from the group consisting of a biological macromolecule, a lipid, a lipid micelle, a lipid monolayer, a lipid bilayer, an artificial membrane, a naturally-occurring biological membrane, a whole cell, a seed, an organ, and a whole organism.

23. The method of claim 22, wherein said biological macromolecule is selected from the group consisting of a polypeptide, a nucleic acid chain, and a polysaccharide.

24. The method of claim 21, wherein said treatment is selected from the group consisting of genetically transforming an organism to co-express an LEA-1 protein with said biological structure; and spraying, immersing, or injecting said biological structure.

25. The method of claim 21, additionally comprising the step of adding one or more additional cryogenic compositions.

26. A method for increasing the resistence of an organism to drought stress,. osmotic stress, heat stress, freezing stress, or a combination thereof, comprising transfecting said organism with an expression vector comprising an isolated GmD-19 DNA sequence operably linked to a promoter which is constitutively or inducibly expressed in said organism.

27. The method of claim 26, wherein said organism is a plant.

28. The method of claim 27, wherein said plant is a cereal plant.

29. The method of claim 28, wherein said cereal plant is selected from the group consisting of wheat, corn, rice, barley, oat, rye, sorghum, and millet.
